(19) United States
(12) Patent Application Publication
(10) Pub. No.: US 2008/0200470 A1
(43) Pub. Date: Aug. 21, 2008

(54) Diaza- or Thiazadione Derivatives With Neuroprotective Activity

(75) Inventors: Maria Luz Lopez-Rodriguez, Madrid (ES); Bellinda Benhamu Salama, Madrid (ES); Joaquin Del Rio Zambrana, Navarra (ES); Diana Frechilla Manso, Navarra (ES)

Correspondence Address: HARNESS, DICKEY, & PIERCE, P.L.C, 7700 Bonhomme, Suite 400, ST. LOUIS, MO, 63105, US

(73) Assignee: Schwartz Pharma S.L

(21) Appl. No.: 10/587,792

(22) PCT Filed: Jan. 28, 2005
(86) PCT No.: PCT/EP2005/000840

§ 371 Date: Aug. 16, 2007

(30) Foreign Application Priority Data
Jan. 30, 2005 (ES) P 200400205

(51) Int. Cl. A61K 31/4985; C07D 487/04; C07D 241/00; C07D 277/02; A61K 31/426; A61K 31/407; A61K 31/437; A61P 25/18; A61P 25/24; A61P 25/06; A61P 25/22

(52) U.S. Cl. 514/249; 548/453; 546/183; 544/349; 548/183; 514/369; 514/413; 514/300

(57) ABSTRACT

The present invention relates to certain derivatives of cycloalkanediones invariably substituted with a chroman-2-yl, 2-quinolyl or —O-phenyl residue which are serotonin (5-hydroxytryptamine, 5-HT) 5-HT_{1A} receptor subtype agonists modulators, to their stereochemical isomers and to their use in the preparation of a medicament for the treatment of pathological states for which an agonist a modulator of these receptors is indicated.

### FIELD OF THE INVENTION

The present invention relates to certain derivatives of cycloalkanediones invariably substituted with a chroman-2-yl, 2-quinolyl or —O-phenyl residue which are serotonin (5-hydroxytryptamine, 5-HT) 5-HT_{1A} receptor subtype agonists modulators, to their stereochemical isomers and to their use in the preparation of a medicament for the treatment of pathological states for which an agonist a modulator of these receptors is indicated.

### BACKGROUND OF THE INVENTION

PCT/ES03/00394 discloses cycloalkanedione derivatives of general formula Ia:

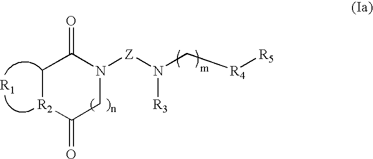

wherein:
R₁ is selected from the group formed by H, —(CH₂)₃—, —(CH₂)₄—, —CH₂—S—CH₂, —S—CH₂—CH₂—;
R₂ is selected from the group formed by N, S;
n has a value of 0 or 1;
Z is selected from the group formed by C₂-C₁₀-alkyl, C₂-C₁₀-alkenyl, C₂-C₁₀-alkinyl;
R₃ is selected from the group formed by H, C₁-C₁₀-alkyl, aryl, aralkyl;
m has a value of 0 to 2;
R₄ is selected from the group formed by O, CH₂;
R₅ is selected from the group formed by:

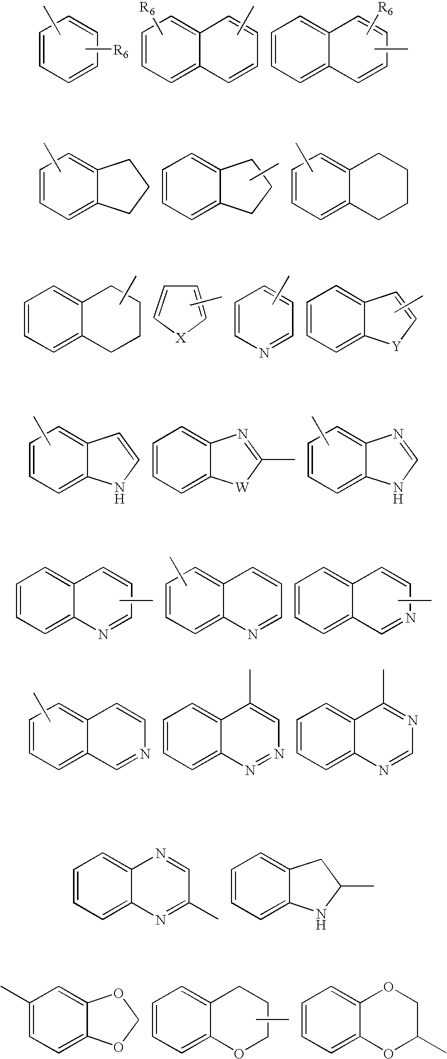

wherein:
R₆ is selected from the group formed by H, C₁-C₅-alkyl, C₁-C₅-alkoxy, OH, F, Cl, Br, I;
X is selected from the group formed by O, S, NH, NCH₃;
Y is selected from the group formed by O, NH;
W is selected from the group formed by S, NH.

PCT/ES03/00394 describes radioligand displacement tests to characterize the in vitro affinity and selectivity in the 5-HT_{1A} cerebral receptors of some of the possible compounds represented by the previous Markush formula (Ia), whilst the functional character (agonist/antagonist) was determined by the study of their effect on adenylate cyclase in HeLa cells transfected with the human 5-HT_{1A} receptor, measuring their inhibiting effect on the stimulation of the enzyme induced by forskolin for the compounds:
2-[4-[(Chroman-2-yl)methylamino]butyl]-1,3-dioxoperhydropyrrolo[1,2-c]imidazole, (a)
2-[4-[(Chroman-2-yl)methylamino]butyl]-1,3-dioxoperhydroimidazo[1,5-b]thiazole, (b)
2-[4-[(Chroman-2-yl)methylamino]butyl]-1,3-dioxoperhydroimidazo[1,5-c]thiazole, (c)
3-[4-[(Chroman-2-yl)methylamino]butyl]-2,4-dioxothiazolidine, (d)
2-[4-[2-(Phenoxy)ethylamino]butyl]-1,3-dioxoperhydropyrrolo[1,2-c]imidazole, (e)

For these compounds (a, b, c, d, e), an in vivo functional characterization test was performed by the quantification of the hypothermia associated to the stimulation of the receptor. Furthermore, the neuroprotective effect was evaluated by in vitro experimental models using primary cultures of rat hippocampus exposed to serum deprivation (compounds a, d, and e), to a toxic concentration of glutamate (compound a), or incubated in conditions of hypoxia and absence of glucose (compound a). On the other hand, the determination of the in vivo neuroprotective action is evaluated both in the transient global ischemia model in gerbils (compounds a and e) and in the permanent focal ischemia model in rats (compound a).

### SUMMARY OF THE INVENTION

The present invention relates to a group of cycloalkanedione derivatives which are invariably substituted with a chroman-2-yl residue, a 2-quinolyl residue or an —O-phenyl residue.

In extensive studies the inventors have surprisingly identified a class of compounds with a high affinity for the 5-HT_{1A} receptor and remarkable neuroprotective properties.

The 5-HT_{1A} affinity has been demonstrated by in vitro radioligand displacement tests. Likewise, their affinity for the serotonergic 5-HT_{2A}, 5-HT₃, 5-HT₄ and 5-HT₇ receptors, 5-HT transporter, adrenergic α₁ and dopaminergic D₂ receptors have been characterized. The functional character (agonist/antagonist) of the new ligands was studied, determining the inhibition of the stimulating effect of forskolin on adenylate cyclase and studying, furthermore, in vivo, the 5-HT_{1A} agonist character of the new compounds by hypothermia analysis. In the same way, the compounds of the present invention have shown in vitro neuroprotective action on primary cultures of rat hippocampus, considering those models of neuronal death (deprivation of trophic factors and deprivation of oxygen and glucose) wherein the serotonergic 5-HT_{1A} agonists are more effective. The protective effect was also studied for cerebral infarction induced by permanent occlusion in the middle cerebral artery in rats.

According to a first aspect of the present invention, it relates to compounds of the general formula I:

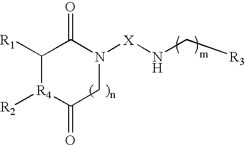

their stereochemically isomer forms, hydrates, solvates and pharmaceutically acceptable salts thereof, wherein:
R₁ and R₂ are H or are methylene groups bound together forming with the heterocyclic ring a 5- or 6-membered ring; if R₄═S then R₁ is H and R₂ is absent;
R₄ is selected from the group consisting of N and S;
n being an integer from 0 to 1;
X is selected from the group consisting of C₂-C₁₀-alkyl, C₂-C₁₀-alkenyl and —CH₂—Y—CH₂—; wherein Y is phenyl;
m being an integer from 1 to 2;
R₃ is selected from the group consisting of chroman-2-yl, 2-quinolyl and —O-phenyl, wherein the aromatic ring of the chromanyl moiety, the quinolyl or the phenyl residue is optionally substituted by one or more groups chosen from C₁-C₆-alkoxy, C₁-C₆-alkyl, halogen, C₂-C₆-alkenyl, halo-(C₁-C₆)-alkyl, halo-(C₁-C₆)-alkoxy, phenyl, phenyl(C₁-C₆)-alkyl, phenoxy, C₁-C₆-alkylcarbonyl, phenylcarbonyl, phenyl(C₁-C₆)alkylcarbonyl, C₁-C₆-alkoxycarbonyl, phenyl(C₁-C₆)alkoxycarbonyl, C₁-C₆-alkylcarbonylamino, hydroxy, cyano, nitro, amino, N—(C₁-C₆)-alkylamino, N,N—(C₁-C₆)-dialkylamino, carboxy, sulfo, sulfamoyl, sulfonylamino, (C₁-C₆)alkylaminosulfonyl or (C₁-C₆)alkylsulfonylamino; or wherein the phenyl ring is substituted by two neighbouring residues, which together with the phenyl ring to which they are attached form tetrahydronaphthyl; wherein each alkyl is optionally substituted with hydroxy or amino;
provided that the compound is not 2-[4-[(chroman-2-yl)methylamino]butyl]-1,3-dioxoperhydropyrrolo[1,2-c]imidazole, 3-[4-[(chroman-2-yl)methylamino]butyl]-2,4-dioxothiazolidine, 3-[5-[(chroman-2-yl)methylamino]pentyl]-2,4-dioxothiazolidine, 3-[6-[(chroman-2-yl)methylamino]hexyl]-2,4-dioxothiazolidine, 2-[4-[2-(phenoxy)ethylamino]butyl]-1,3-dioxoperhydropyrrolo[1,2-c]imidazole or 3-[4-[2-(phenoxy)ethylamino]butyl]-2,4-dioxothiazolidine.

In a preferred embodiment, R₃ is preferably selected from the group consisting of chroman-2-yl, 2-quinolyl and —O-phenyl, wherein the phenyl residue is optionally substituted by a group chosen from C₁-C₆-alkoxy, C₁-C₆-alkyl, or halogen.

The present invention comprises three main embodiments:

(1) m is 1 and R₃ is optionally substituted chroman-2-yl

(2) m is 2 and R₃ is optionally substituted O-phenyl

(3) m is 1 and R₃ is optionally substituted 2-quinolyl

According to a first preferred main embodiment of the present invention, m is 1 and R₃ is chroman-2-yl, the phenyl ring of which is unsubstituted or substituted by one or more groups chosen from C₁-C₆-alkoxy, C₁-C₆-alkyl, halogen, C₂-C₆-alkenyl, halo-(C₁-C₆)-alkyl, halo-(C₁-C₆)-alkoxy, phenyl, phenyl(C₁-C₆)-alkyl, phenoxy, C₁-C₆-alkylcarbonyl, phenylcarbonyl, phenyl(C₁-C₆)alkylcarbonyl, C₁-C₆-alkoxycarbonyl, phenyl(C₁-C₆)alkoxycarbonyl, C₁-C₆-alkylcarbonylamino, hydroxy, cyano, nitro, amino, N—(C₁-C₆)-alkylamino, N,N—(C₁-C₆)-dialkylamino, carboxy, sulfo, sulfamoyl, sulfonylamino, (C₁-C₆)alkylaminosulfonylor (C₁-C₆)alkylsulfonylamino; wherein each alkyl is optionally substituted with hydroxy or amino. R₃ is preferably unsubstituted chroman-2-yl.

Unless specifically mentioned otherwise the term “chroman-2-yl” refers to an unsubstituted chroman-2-yl residue.

According to a first embodiment of this first preferred main embodiment of the invention, R₁ and R₂ are methylene groups bound together forming with the heterocyclic ring a 5- or 6-membered ring and R₄ is N.

Those compounds wherein m is 1 and R₃ is chroman-2-yl, R₁ and R₂ are methylene groups bound together forming with the heterocyclic ring a 5- or 6-membered ring; R₄ is N; and X is selected from the group consisting of C₂-C₁₀-alkyl, (E)-2-butenyl, 3-methylbenzyl or 4-methylbenzyl are preferred.

In a second embodiment of this first preferred main embodiment of the present invention, R₁ is H; R₂ is absent; R₄ is S; m is 1; R₃ is chroman-2-yl; and X is selected from the group consisting of C₂-C₁₀-alkyl, C₂-C₁₀-alkenyl, or —CH₂—Y—CH₂—, wherein Y is phenyl. In one embodiment n is preferably 0.

In a more preferred embodiment of the present invention, it provides compounds of formula (I) wherein: R₁ is H; R₂ is absent; R₄ is S; m is 1; R₃ is chroman-2-yl; and X is C₂-C₁₀-alkyl. In one embodiment n is preferably 0.

A second preferred main embodiment of the invention relates to compounds wherein m is 2 and R₃ is —O-phenyl optionally substituted by one or more groups chosen from C₁-C₆-alkoxy, C₁-C₆-alkyl, halogen, C₂-C₆-alkenyl, halo-(C₁-C₆)-alkyl, halo-(C₁-C₆)-alkoxy, phenyl, phenyl(C₁-C₆)-alkyl, phenoxy, C₁-C₆-alkylcarbonyl, phenylcarbonyl, phenyl(C₁-C₆)alkylcarbonyl, C₁-C₆-alkoxycarbonyl, phenyl(C₁-C₆)alkoxycarbonyl, C₁-C₆-alkylcarbonylamino, hydroxy, cyano, nitro, amino, N—(C₁-C₆)-alkylamino, N,N—(C₁-C₆)-dialkylamino, carboxy, sulfo, sulfamoyl, sulfonylamino, (C₁-C₆)alkylaminosulfonyl or (C₁-C₆)alkylsulfonylamino; or wherein the phenyl ring is substituted by two neighbouring residues, which together with the phenyl ring to which they are attached form tetrahydronaphthyl; wherein each alkyl is optionally substituted with hydroxy or amino.

According to a more preferred embodiment of the second main embodiment of the invention, it relates to compounds of formula (I) wherein: m=2 and R₃ is —O-phenyl, wherein the phenyl ring is substituted by one or more groups chosen from phenyl, C₁-C₆-alkoxycarbonyl, C₁-C₆-alkylcarbonylamino, C₁-C₆-alkoxy, C₁-C₆-alkyl, halo-(C₁-C₆)-alkyl, or halogen or wherein the phenyl group is substituted by two neighbouring residues, which together with the phenyl group to which they are attached form tetrahydronaphthyl.

In another preferred embodiment, m=2 and R₃ is —O-phenyl, wherein the phenyl ring is substituted by one or more groups chosen from C₁-C₆-alkoxy, C₁-C₆-alkyl, or halogen.

Most preferred compounds are those wherein the phenyl residue is optionally substituted by one or more groups chosen from methoxy, ethoxy, propoxy, isopropoxy, ethyl, propyl, isopropyl, bromide, trifluoromethyl, methylamide or ethoxycarbonyl.

Particularly preferred are those compounds wherein the phenyl residue is substituted in ortho and/or meta position.

According to a preferred embodiment of this second preferred embodiment of the invention, R₁ and R₂ are methylene groups bound together forming with the heterocyclic ring a 5- or 6-membered ring; and R₄ is N.

Particularly preferred compounds are those wherein R₁ and R₂ are methylene groups bound together forming with the heterocyclic ring a 5-membered ring; R₄ is N; n is 0; X is C₂-C₁₀-alkyl; m is 2; R₃ is —O-phenyl optionally substituted by one or more groups chosen from phenyl, C₁-C₆-alkoxycarbonyl, C₁-C₆-alkylcarbonylamino, C₁-C₆-alkoxy, C₁-C₆-alkyl, halo-(C₁-C₆)-alkyl, or halogen or wherein the phenyl group is substituted by two neighbouring residues, which together with the phenyl group to which they are attached form tetrahydronaphthyl.

In a more specific embodiment R₃ is O-phenyl substituted by a group chosen from C₁-C₆-alkoxy, C₁-C₆-alkyl, or halogen.

In another preferred embodiment of this second preferred main embodiment of the invention, R₁ is H, R₂ is absent and R₄ is S. Particularly those wherein X is C₂-C₁₀-alkyl and n is 0.

According to a third main embodiment of the present invention, it relates to compounds of formula (I) wherein m is 1 and R₃ is 2-quinolyl, the aromatic ring of which is unsubstituted or substituted by one or more groups chosen from C₁-C₆-alkoxy, C₁-C₆-alkyl, halogen, C₂-C₆-alkenyl, halo-(C₁-C₆)-alkyl, halo-(C₁-C₆)-alkoxy, phenyl, phenyl(C₁-C₆)-alkyl, phenoxy, C₁-C₆-alkylcarbonyl, phenylcarbonyl, phenyl(C₁-C₆)alkylcarbonyl, C₁-C₆-alkoxycarbonyl, phenyl(C₁-C₆)alkoxycarbonyl, C₁-C₆-alkylcarbonylamino, hydroxy, cyano, nitro, amino, N—(C₁-C₆)-alkylamino, N,N—(C₁-C₆)-dialkylamino, carboxy, sulfo, sulfamoyl, sulfonylamino, (C₁-C₆)alkylaminosulfonyl or (C₁-C₆)alkylsulfonylamino; wherein each alkyl is optionally substituted with hydroxy or amino. R₃ is preferably unsubstituted 2-quinolyl.

Unless specifically mentioned otherwise the term “2-quinolyl” refers to an unsubstituted quinolyl residue.

In a preferred embodiment of this third preferred main embodiment of the invention, R₁ and R₂ are methylene groups bound together forming with the heterocyclic ring a 5- or 6-membered ring; and R₄ is N. Those compounds wherein n is 0; and X is C₂-C₁₀-alkyl are particularly preferred.

In the context of the present invention, the term “alkyl” relates to a saturated, linear or branched hydrocarbon chain. The “alkyl”-group may be unsubstituted or substituted. “Alkyl” is preferably unsubstituted. If the “alkyl” group (also as a part of e.g. phenylalkyl, alkylcarbonyl or alkoxy) is substituted, the substituents are preferably hydroxyl or amino. Unless specifically mentioned otherwise, the term “alkyl” refers to an unsubstituted hydrocarbon chain.

In the context of the present invention, the term “C₂-C₁₀-alkyl” relates to a saturated, linear or branched hydrocarbon chain, that contains from 2 to 10 carbon atoms. The term “C₂-C₁₀-alkenyl” relates to a linear or branched hydrocarbon chain that contains from 2 to 10 carbon atoms and which has at least one double bond.

The term “C₁-C₆-alkyl” relates to a saturated, linear or branched hydrocarbon chain that contains from 1 to 6 carbon atoms.

The term “halogen”, as used in this specification, consisting of fluorine, chloride, bromide, and iodine.

The term “halo-(C₁-C₆)-alkyl” refers to “C₁-C₆ alkyl” as defined above, which is substituted with at least one halogen atom. It includes as preferred embodiments difluoromethyl and trifluoromethyl.

The term “(C₁-C₆)-alkoxy” refers to the group —O—(C₁-C₆)-alkyl.

The term “halo-(C₁-C₆)-alkoxy” refers to “C₁-C₆ alkoxy” as defined above, which is substituted with at least one halogen atom. It includes as preferred embodiments difluoromethoxy and trifluoromethoxy.

The term 5-HT_{1A} receptor “modulator” as used herein includes pure and partial agonists as well as antagonists of the serotonin 5-HT_{1A} receptor. Preferred are “agonists”, i.e. compounds with at least partial agonistic activity at the 5-HT_{1A} receptor.

The compounds of the present invention can include enantiomers depending on their asymmetry or diastereoisomers. It is also possible stereoisomerism with regard to double bounds, thereby in some cases the molecule can exist as the (E) isomer or the (Z) isomer. Each of the different possible enantiomers, diastereoisomers or isomers with regard to double bounds and the mixtures thereof, their racemic and optically pure forms are included in the scope of the present invention.

Optically pure isomers may be prepared using chiral synthons or chiral reagents, or resolved using conventional techniques.

The expression stereochemically isomeric forms, as used in this specification, defines all the possible isomeric forms wherein the compounds of formula (I) can be present. Unless otherwise mentioned or indicated, the chemical name of the compounds designates the mixture of all the possible stereochemically isomeric forms, said mixtures containing all the diastereoisomers and enantiomers of the basic molecular structure.

When used hereinafter in this specification, the expression compounds of formula (I) has the object of also including the pharmaceutically acceptable acid addition salts and all the stereoisomeric forms.

The pharmaceutically acceptable acid addition salts previously mentioned in this specification have the object of comprising the acid addition salts that can be conveniently obtained by treatment of the base form of the compounds of formula (I) with appropriate inorganic acids such as hydrochloride or hydrobromic acids, sulphuric, nitric, phosphoric acid and analogous acids; or organic acids, such as, e.g. acetic, hydroxyacetic, propionic, lactic, pyruvic, oxalic, malonic, succinic, maleic, fumaric, malic, tartaric, citric, methanesulphonic, ethanesulphonic, benzenesulphonic, p-toluenesulphonic, cyclamic, salicylic, p-aminosalicylic, palmoic acids and analogues. Inversely, said forms of acid addition salts can become the free base forms due to treatment with an appropriate base.

The expression acid “addition salt” comprises amorphous as well as crystalline salts and also comprises the hydrates and the forms of solvent addition that the compounds of formula (I) may form. Examples of said forms are hydrates, alcoholates and analogues.

In the field of the present invention physiologically compatible salts will be preferable.

General method of preparation of the compounds of the present invention:

A solution of 1.3 mmol of corresponding halogenated derivative dissolved in 5 mL of dry acetonitrile is added dropwise to 2.0 mmol of the corresponding alkylamine, dissolved in 2 mL of dry acetonitrile. The reaction mixture is heated to 60° C. with stirring for 4-6 hours (t.l.c.). After cooling, the solvent is removed at reduced pressure, the residue is dissolved in methylene chloride (25 mL) and is washed with an aqueous solution of 20% potassium carbonate. Then, the organic phase is dried over anhydrous Na₂SO₄ and the solvent is removed at reduced pressure. The resulting oil is purified by silica gel column chromatography in the appropriate solvent mixture, producing the final product as a free base. The compound is transformed to its hydrochloride and is purified by recrystallization.

The final products have been structurally characterized by IR, NMR and quantitative elemental analysis techniques. For greater ease of handling, when the final product is not crystalline, it is transformed in a pharmaceutically acceptable salt, derived from an inorganic or organic acid.

Preferred compounds of the present invention are:
(a) 2-[4-[(Chroman-2(R)-yl)methylamino]butyl]-1,3-dioxoperhydropyrrolo[1,2-c]imidazole;
(b) 2-[4-[(Chroman-2-yl)methylamino]butyl]-1,3-dioxoperhydroimidazo[1,5-a]pyridine;
(c) 2-[4-[(Chroman-2-yl)methylamino]butyl]-1,3-dioxoperhydropyrrolo[1,2-a]pyrazine;
(d) 2-[5-[(Chroman-2-yl)methylamino]pentyl]-1,3-dioxoperhydropyrrolo[1,2-c]imidazole;
(e) 2-[6-[(Chroman-2-yl)methylamino]hexyl]-1,3-dioxoperhydropyrrolo[1,2-c]imidazole;
(f) 2-[3-[(Chroman-2-yl)methylamino]propyl]-1,3-dioxoperhydropyrrolo[1,2-c]imidazole;
(g) 3-[8-[(Chroman-2-yl)methylamino]octyl]-2,4-dioxothiazolidine;
(h) 2-[4-[(Chroman-2(S)-yl)methylamino]butyl]-1,3-dioxoperhydropyrrolo[1,2-c]imidazole;
(i) 2-[8-[(Chroman-2-yl)methylamino]octyl]-1,3-dioxoperhydropyrrolo[1,2-c]imidazole;
(j) 2-[3-[[(Chroman-2-yl)methylamino]methyl]benzyl]-1,3-dioxoperhydropyrrolo[1,2-c]imidazole;
(k) 2-[4-[[(Chroman-2-yl)methylamino]methyl]benzyl]-1,3-dioxoperhyd(k) ropyrrolo[1,2-c]imidazole;
(l) (E)-2-[4-[(Chroman-2-yl)methylamino]but-2-enyl]-1,3-dioxoperhydropyrrolo[1,2-c]imidazole;
(m) 2-[4-[2-(o-Methoxyphenoxy)ethylamino]butyl]-1,3-dioxoperhydropyrrolo[1,2-c]imidazole;
(n) 2-[4-[2-(m-Methoxyphenoxy)ethylamino]butyl]-1,3-dioxoperhydropyrrolo[1,2-c]imidazole;
(o) 2-[4-[2-(o-Bromophenoxy)ethylamino]butyl]-1,3-dioxoperhydropyrrolo[1,2-c]imidazole;
(p) 2-[4-[2-(m-Bromophenoxy)ethylamino]butyl]-1,3-dioxoperhydropyrrolo[1,2-c]imidazole;
(q) 2-[4-[2-(o-Ethylphenoxy)ethylamino]butyl]-1,3-dioxoperhydropyrrolo[1,2-c]imidazole;
(r) 2-[4-[2-(m-Ethylphenoxy)ethylamino]butyl]-1,3-dioxoperhydropyrrolo[1,2-c]imidazole;
(s) 2-[4-[2-(o-Isopropylphenoxy)ethylamino]butyl]-1,3-dioxoperhydropyrrolo[1,2-c]imidazole;
(t) 2-[4-[(2-quinolyl)methylamino]butyl]-1,3-dioxoperhydropyrrolo[1,2-c]imidazole;
(u) 2-[4-[2-(o-Ethoxyphenoxy)ethylamino]butyl]-1,3-dioxoperhydropyrrolo[1,2-c]imidazole;
(v) 2-[4-[2-(o-Isopropoxyphenoxy)ethylamino]butyl]-1,3-dioxoperhydropyrrolo[1,2-c]imidazole;
(w) 2-[4-[2-[m-(Trifluoromethyl)phenoxy]ethylamino]butyl]-1,3-dioxoperhydropyrrolo[1,2-c]imidazole;
(x) 2-[4-[2-(1,1′-Biphenyl-2-yloxy)ethylamino]butyl]-1,3-dioxoperhydropyrrolo[1,2-c]imidazole;
(y) 2-[4-[2-[o-(Acetylamino)phenoxy]ethylamino]butyl]-1,3-dioxoperhydropyrrolo[1,2-c]imidazole;
(z) 2-[4-[2-[m-(Acetylamino)phenoxy]ethylamino]butyl]-1,3-dioxoperhydropyrrolo[1,2-c]imidazole;
(aa) 2-[4-[2-[o-(Ethoxycarbonyl)phenoxy]ethylamino]butyl]-1,3-dioxoperhydropyrrolo[1,2-c]imidazole;
(bb) 2-[4-[2-(5,6,7,8-tetrahydronaphth-1-yloxy)ethylamino]butyl]-1,3-dioxoperhydropyrrolo[1,2-c]imidazole;
(cc) 2-[4-[2-(2,3-Dimethylphenoxy)ethylamino]butyl]-1,3-dioxoperhydropyrrolo[1,2-c]imidazole;
(dd) 2-[4-[(Chroman-2-yl)methylamino]butyl]-1,4-dioxoperhydropyrido[1,2-a]pyrazine;
(ee) (Z)-2-[4-[(Chroman-2-yl)methylamino]but-2-enyl]-1,4-dioxoperhydropyrrolo[1,2-c]imidazole;
(ff) 3-[4-[2-(o-Ethoxyphenoxy)ethylamino]butyl]-2,4-dioxothiazolidine;
(gg) 3-[6-[2-(o-Ethoxyphenoxy)ethylamino]hexyl]-2,4-dioxothiazolidine;
(hh) 3-[8-[2-(o-Ethoxyphenoxy)ethylamino]octyl]-2,4-dioxothiazolidine;
(ii) 2-[4-[2-(o-Ethoxyphenoxy)ethylamino]butyl]-1,3-dioxoperhydroimidazo[1,5-a]pyridine;
(jj) 2-[6-[2-(o-Ethoxyphenoxy)ethylamino]hexyl]-1,3-dioxoperhydroimidazo[1,5-a]pyridine;
(kk) 2-[4-[(2-Quinolyl)methylamino]butyl]-1,3-dioxoperhydroimidazo[1,5-a]pyridine;
(ll) 2-[6-[(2-Quinolyl)methylamino]hexyl]-1,3-dioxoperhydroimidazo[1,5-a]pyridine;
   their stereochemically isomer forms, hydrates, solvates and pharmaceutically acceptable salts thereof.

The cellular death produced by oxygen and glucose deprivation in primary cultures of rat hippocampal neurons is a model that has a much closer similarity with cerebral infarction than the cellular death caused by serum deprivation in the culture medium. Whilst in this last model, the death, of an apoptotic nature, takes place due to the elimination of the trophic factors from the medium, oxygen and glucose deprivation causes a death with similar characteristics to that which takes place in an ischemic stroke. In accordance with the predictive value of these in vitro studies, the compound (a) of PCT/ES03/00394 only exercises a protective effect against cerebral infarction induced by permanent occlusion of the middle cerebral artery in rats at a dose of 2 mg/kg. On the other hand, as is indicated further on in the present specification, compound (e) disclosed herein, with a neuroprotective effect equal to (−)-BAYx3702 and about four times greater than the compound (a) of the previous document against death due to anoxia, significantly reduces the volume of cortical infarction in the same focal ischemia model in the rat at a much lower accumulated dose, 0.04 mg/kg, similar to the effective dose of (−)-BAYx3702 in this model.

Taking into account its 5-HT_{1A} receptor affinity and its neuroprotective capacity, the compounds of formula (I) are useful in the treatment and/or prevention of pathological states wherein the 5-HT_{1A} receptor modulators and particularly agonists are indicated, such as, for example, the treatment and/or prophylaxis of cerebral damage caused by thromboembolic stroke or traumatic brain damage, as well as the treatment and/or prevention of Parkinson's disease, depression including particularly endogenous “major” depression, migraine, pain, psychosis such as e.g. schizophrenia; mood disorders, such as anxiety disorders (e.g. obsessional compulsive disorders, generalised anxiety) and aggressive disorders (including mixed aggressive-anxiety/depressive disorders); urinary tract disorders, in particular urinary incontinence, e.g. stress incontinence.

Therefore, according to a second aspect of the present invention, it relates to a pharmaceutical composition that comprises a therapeutically effective quantity of any of the compounds of formula (I) together with a pharmaceutically acceptable carrier.

A third aspect of the present invention relates to the use of compounds of formula (I) in the manufacture of a medicament for the treatment and/or prophylaxis of Parkinson's disease, of the cerebral damage caused by thromboembolic stroke or traumatic brain damage, depression, migraine, and/or pain, psychosis (e.g. schizophrenia); mood disorders, such as anxiety disorders (e.g. obsessional compulsive disorders, generalised anxiety) and aggressive disorders (including mixed aggressive-anxiety/depressive disorders); urinary tract disorders (e.g. incontinence).

This third aspect may alternatively be formulated as a method for treatment of the diseases mentioned above in a human comprising administering to a human in need thereof an effective amount of pharmaceutical product as described herein.

For ease of administration, the compounds of the present invention can be formulated in various pharmaceutical forms. As appropriate compositions, one can cite all the compositions usually used for drugs administered systemically or locally and externally. To prepare the pharmaceutical compositions of this invention, a therapeutically effective quantity of the particular compound, optionally in the form of an acid addition salt, as an active ingredient, is combined in an intimate mixture with a pharmaceutically acceptable carrier, which can have a large variety of forms, depending on the form of preparation desired to be administered. These pharmaceutical compositions are desirably found in the form of an appropriate unit dose, preferably for oral or rectal administration or by parenteral injection.

For example, in the preparation of the compositions in the form of an oral dose, any of the usual pharmaceutically acceptable carrier can be used, such as, e.g. water buffered and/or isotonic aqueous solutions, glycols, oils, alcohols and analogues in the case of liquid oral preparations such as suspensions, syrups, elixirs and solutions; or solid carriers such as starches, sugars, kaolin, lubricants, ligands, disintegrating agents and analogues, in the case of powders, pills, capsules and tablets.

Due to their ease of administration, tablets and capsules represent the most advantageous oral unit dose form, in which case solid pharmaceutical carriers are evidently used. For parenteral compositions, the carrier will usually comprise sterile water, at least in large part, although other ingredients can be included, e.g. to favour solubility. Injectable solutions, for example, can be prepared wherein the carrier comprises saline solution, glucose solution or a mixture of saline solution and glucose solution. Also, if suitable the compounds of the present invention may be also administered transdermally.

The present invention is illustrated with the following non-limiting examples.

### EXAMPLES

### Example 1

### 2-[4-[(Chroman-2(R)-yl)methylamino]butyl]-1,3-dioxoperhydropyrrolo[1,2-c]imidazole, (diastereoisomers) (a)

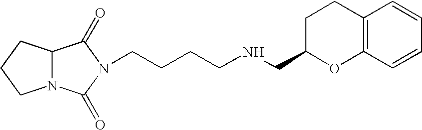

Chromatography: ethyl acetate.

Yield: 35%.

¹H-NMR (CDCl₃, δ): 1.47-1.86 (m, 5H, —(CH₂)₂—, H₇), 1.91-2.12 (m, 4H, 2H₃, 2H₆), 2.16-2.34 (m, 1H, H₇), 2.64-2.92 (m, 6H, 2CH₂NH, 2H_{4′}), 3.16-3.28 (m, 1H, H₅), 3.48 (t, J=7.1 Hz, 2H, NCH₂), 3.66 (dt, J=11.2, 7.3 Hz, 1H, H₅), 4.05 (dd, J=9.1, 7.3 Hz, 1H, H₇ₐ), 4.11-4.18 (m, 1H, H_{2′}), 6.81 (t, J=7.6 Hz, 2H, H_{6′}, H_{8′}), 7.00-7.10 (m, 2H, H_{5′}, H_{7′}).

¹³C-NMR (CDCl₃, δ): 24.6 (C₃), 25.6 (C₄), 25.8 (CH₂), 26.9 (CH₂), 27.1 (C₆), 27.5 (C₇), 38.7 (NCH₂), 45.4 (C₅), 49.3 (CH₂CH₂NH), 54.1 (HNCH₂CH), 63.2 (C₇ₐ), 75.0 (C₂), 116.7 (C_{8′}), 120.1 (C_{6′}), 121.9 (C_{4′a}), 127.1 (C_{7′}), 129.4 (C_{5′}), 154.5 (C_{8′a}), 160.8 (C₃), 173.9 (C₁).

Analysis calculated for C₂₀H₂₇N₃O₃.HCl:

C, 60.98; H, 7.16; N, 10.67

Found: C, 60.15; H, 7.14; N, 10.45

### Example 2

### 2-[4-[(Chroman-2-yl)methylamino]butyl]-1,3-dioxoperhydroimidazo[1,5-a]pyridine, (b)

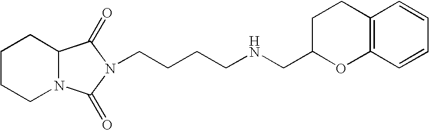

Chromatography: ethyl acetate.

Yield: 30%.

¹H-NMR (CDCl₃, 8): 1.06-1.40 (m, 3H, H₆ₐₓ, H₇ₐₓ, H₈ₐₓ), 1.60-1.62 (m, 7H, H_{6ec}, (CH₂)₂—, 2H_{3′}), 1.88-2.09 (m, 1H, H_{7ec}), 2.11-2.18 (m, 1H, H_{8ec}), 2.71-2.74 (m, 4H, 2NHCH₂), 2.85-2.87 (m, 3H, H₅ₐₓ, 2H_{4′}), 3.47 (t, 2H, J=6.6 Hz, NCH₂), 3.67 (dd, 1H, J=11.9, 4.3 Hz, H₈ₐ), 4.03-4.14 (m, 2H, H_{5ec}, H_{2′}), 6.76 (t, 2H, J=7.6 Hz, H_{6′}, H_{8′}), 6.98 (t, 2H, J=6.3 Hz, H_{5′}, H_{7′}).

Analysis calculated for C₂₁H₂₉N₃₃.HCl.H₂O:

C, 59.21; H, 7.57; N, 9.87

Found: C, 58.76; H, 7.01; N, 9.89

### Example 3

### 2-[4-[(Chroman-2-yl)methylamino]butyl]-1,3-dioxoperhydropyrrolo[1,2-a]pyrazine, (c)

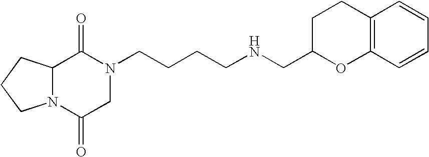

Chromatography: ethyl acetate.

Yield: 35%.

¹H-NMR (CDCl₃, δ): 1.14-2.09 (m, 9H, —(CH₂)₂—, 2H₇, H₈, 2H_{3′}), 2.28-2.34 (m, 1H, H₈), 2.65-2.93 (m, 6H, 2NHCH₂, 2H_{4′}), 3.29-3.56 (m, 4H, NCH₂, 2H₆), 3.71 (d, 1H, J=11.9 Hz, H₃), 4.04-4.14 (m, 3H, H₃, H₈ₐ, H_{2′}), 6.67-6.80 (m, 2H, H_{6′}, H_{8′}), 6.95-7.22 (m, 2H, H_{5′}, H_{7′}).

¹³C-NMR (CDCl₃, δ): 22.8 (C₇), 24.7, 25.0, 25.7, 26.7, 29.0 (—(CH₂)₂—, C₈, C_{3′}, C_{4′}), 45.4 (NCH₂), 46.0 (C₆), 49.4 (NHCH₂), 51.9 (C₃), 54.0 (NHCH₂), 59.2 (C₈ₐ), 74.7 (C_{2′}), 116.9 (C_{8′}), 120.4 (C_{6′}), 122.1 (C_{4a′}), 127.4 (C_{7′}), 129.7 (C_{5′}), 154.6 (C_{8a′}), 163.4 (C₄), 167.4 (C₁).

Analysis calculated for C₂₁H₂₉N₃O₃.HCl.2H₂O:

C, 56.81; H, 7.72; N, 9.46

Found: C, 56.73; H, 7.09; N, 9.55

### Example 4

### 2-[5-[(Chroman-2-yl)methylamino]pentyl]-1,3-dioxoperhydropyrrolo[1,2-c]imidazole, (d)

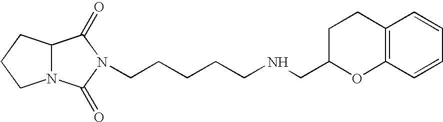

Chromatography: ethyl acetate.

Yield: 32%

¹H-NMR (CDCl₃, δ): 1.34-1.39 (m, 2H, —(CH₂)—), 1.61-1.76 (m, 6H, —(CH₂)₂—, 2H_{3′}), 2.01-2.10 (m, 3H, 2H₆, H₇), 2.17-2.33 (m, 1H, H₇), 2.75-2.78 (m, 4H, CH₂CH₂NH, HNCH₂CH), 2.81-2.94 (m, 2H, 2H_{4′}), 2.93-2.98 (m, 1H, H₅), 3.45 (t, J=7.1 Hz, 2H, NCH₂), 3.58-3.78 (m, 1H, H₅), 4.06 (dd, J=9.1, 7.3 Hz, 1H, H₇ₐ), 4.29-4.39 (m, 1H, H_{2′}), 6.82-6.89 (m, 2H, H_{6′}, H_{8′}), 7.02-7.11 (m, 2H, H_{5′}, H_{7′}).

¹³C-NMR (CDCl₃, δ): 23.6 (CH₂), 24.9 (C_{3′}), 25.1 (C_{4′}), 26.9 ((CH₂)₂), 27.2 (C₆), 27.4 (C₇), 38.0 (NCH₂), 38.5 (C₅), 45.3 (CH₂CH₂NH), 47.9 (HNCH₂CH), 63.3 (C₇ₐ), 70.9 (C₂), 117.1 (C_{8′}), 121.0 (C_{6′}), 121.2 (C_{4′a}), 127.4 (C_{5′}), 129.3 (C_{7′}), 153.0 (C_{8′a}), 160.7 (C₃), 173.9 (C₁).

Analysis calculated for C₂₁H₂₉N₃O₃.HCl.H₂O:

C, 59.21; H, 7.57; N, 9.87

Found: C, 59.19; H, 7.17; N, 9.64

### Example 5

### 2-[6-[(Chroman-2-yl)methylamino]hexyl]-1,3-dioxoperhydropyrrolo[1,2-c]imidazole, (e)

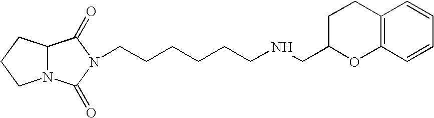

Chromatography: chloroform/methanol, 9.5:0.5.

Yield: 35%.

¹H-NMR (CDCl₃, δ): 1.28-1.35 (m, 4H, —(CH₂)₂—), 1.60-1.80 (m, 6H, —(CH₂)₂—, 2H_{3′}), 1.96-2.14 (m, 3H, 2H₆, H₇), 2.17-2.33 (m, 1H, H₇), 2.77-3.03 (m, 6H, CH₂CH₂NH, HNCH₂CH, 2H_{4′}), 3.17-3.30 (m, 1H, H₅), 3.45 (t, J=7.1 Hz, 2H, NCH₂), 3.58-3.78 (m, 1H, H₅), 4.06 (dd, J=9.1, 7.3 Hz, 1H, H₇ₐ), 4.29-4.39 (m, 1H, H_{2′}), 6.80-6.93 (m, 2H, H_{6′}, H_{8′}), 7.00-7.08 (m, 2H, H_{5′}, H_{7′}).

¹³C-NMR (CDCl₃δ): 24.1 (CH₂), 24.3 (C_{3′}), 25.5 (C_{4′}), 26.3, 26.5, 27.0 ((CH₂)₃), 27.5 (C₆), 27.8 (C₇), 38.7 (NCH₂), 45.5 (C₅), 49.2 (CH₂CH₂NH), 53.1 (HNCH₂CH), 63.3 (C₇ₐ), 72.7 (C_{2′}), 116.9 (C₈), 120.4 (C_{6′}), 121.7 (C_{4′a}), 127.3 (C_{5′}), 129.5 (C_{7′}), 154.1 (C_{8′a}), 160.9 (C₃), 174.0 (C₁).

Analysis calculated for C₂H₃O₃.HCl.H₂:

C, 60.06; H, 7.79; N, 9.55

Found: C, 60.46; H, 7.41; N, 9.54

### Example 6

### 2-[3-[(Chroman-2-yl)methylamino]propyl]-1,3-dioxoperhydropyrrolo[1,2-c]imidazole, (f)

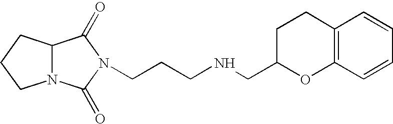

Chromatography: ethyl acetate.

Yield: 40%.

¹H-NMR (CDCl₃, δ): 1.64-2.32 (m, 8H, —(CH₂)—, 2H₆, 2H₇, 2H_{3′}), 2.68-2.88 (m, 6H, 2CH₂NH, 2H_{4′}), 3.18-3.30 (m, 1H, H₅), 3.58 (t, 2H, J=6.8 Hz, NCH₂), 3.65-3.70 (m, 1H, H₅), 4.03-4.17 (m, 2H, H₇ₐ, H_{2′}), 6.79-6.86 (m, 2H, H_{6′}, H_{8′}), 7.02-7.11 (m, 2H, H_{5′}, H_{7′}).

¹³C-NMR (CDCl₃, δ): 24.6 (C_{3′}), 25.6 (C_{4′}), 26.9 (CH₂), 27.5 (C₆), 28.1 (C₇), 36.9 (NCH₂), 45.5 (C₅), 46.9 (CH₂CH₂NH), 54.0 (HNCH₂CH), 63.3 (C₇ₐ), 74.9 (C₂), 116.8 (C_{8′}), 120.2 (C_{6′}), 122.0 (C_{4′a}), 127.2 (C_{7′}), 129.5 (C_{5′}), 154.6 (C_{8′a}), 160.9 (C₃), 174.0 (C₁).

Analysis calculated for C₁₉H₂₅N₃O₃.HCl:

C, 60.07; H, 6.90; N, 11.06

Found: C, 59.65; H, 6.91; N, 10.55

### Example 7

### 3-[8-[(Chroman-2-yl)methylamino]octyl]-2,4-dioxothiazolidine, (g)

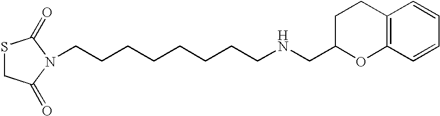

Chromatography: ethyl acetate.

Yield: 35%; m.p. 108-111° C.

¹H-NMR (CDCl₃, δ): 1.29-1.31 (m, 8H, —(CH₂)₄—), 1.55-1.66 (m, 4H, CH₂, 2H_{3′}), 1.71-1.86 (m, 2H, CH₂), 2.70-2.93 (m, 6H, 2NHCH₂, 2H_{4′}), 3.60 (t, J=7.6 Hz, 2H, NCH₂), 3.94 (s, 2H, 2H₅), 4.19-4.25 (m, 2H, H_{2′}, NH), 6.80-6.86 (m, 2H, H_{6′}, H_{8′}), 7.01-7.11 (m, 2H, H_{5′}, H_{7′}).

¹³C-NMR (CDCl₃, δ): 24.3, 25.4, 26.4, 26.9, 27.3, 28.8, 28.9, 29.0 (—(CH₂)₆—, C_{3′}, C_{4′}), 33.5 (C₅), 41.9 (NCH₂), 49.3, 53.3 (2CH₂NH), 74.0 (C_{2′}), 116.6 (C_{8′}), 120.2 (C_{6′}), 121.7 (C_{4a′}), 127.1 (C_{7′}), 129.3 (C_{5′}), 154.2 (C_{8a′}), 171.3, 171.7 (C₂, C₄).

Analysis calculated for C₂₁H₃₀N₂O₃S.HCl.3H₂O:

C, 52.43; H, 7.75; N, 5.82

Found: C, 52.33; H, 6.78; N, 5.79

### Example 8

### 2-[4-[(Chroman-2(S)-yl)methylamino]butyl]-1,3-dioxoperhydropyrrolo[1,2-c]imidazole, (diastereoisomers) (h)

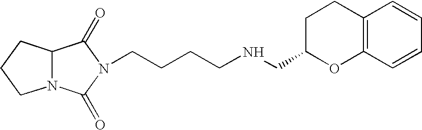

Chromatography: ethyl acetate.

Yield: 38%.

[α]²⁵_{D}=+65 (c=0.5, CHCl₃).

Analysis calculated for C₂₁H₃₀N₂O₃S.HCl.3H₂O:

C, 53.62; H, 7.65; N, 9.38

Found: C, 53.45; H, 7.34; N, 9.45

### Example 9

### 2-[8-[(Chroman-2-yl)methylamino]octyl]-1,3-dioxoperhydropyrrolo[1,2-c]imidazole, (i)

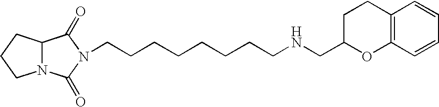

Chromatography: ethyl acetate.

Yield: 35%.

¹H-RMN (CDCl₃, δ): 1.29-1.31 (m, 8H, —(CH₂)₄—), 1.55-1.88 (m, 7H, —(CH₂)₂—, 2H_{3′}, H₇), 1.94-2.34 (m, 3H, 2H₆, H₇), 2.54 (br s, 1H, NH), 2.66-2.97 (m, 6H, 2CH₂NH, 2H_{4′}), 3.18-3.29 (m, 1H, H₅), 3.44 (t, 2H, J=7.3 Hz, NCH₂), 3.62-3.74 (m, 1H, H₅), 4.06 (dd, 1H, J=7.8, 7.6 Hz, H₇ₐ), 4.14-4.21 (m, 1H, H_{2′}), 6.80-6.86 (m, 2H, H_{6′}, H_{8′}), 7.01-7.11 (m, 2H, H_{5′}, H_{7′}).

¹³C-RMN (CDCl₃, δ): 24.6, 25.7, 26.6, 27.0, 27.1, 27.6, 27.9, 29.0 (—(CH₂)₆—, C_{3′}, C_{4′}), 29.3 (C₆), 29.6 (C₇), 39.0 (NCH₂), 45.5 (C₅), 49.8 (CH₂CH₂NH), 54.0 (HNCH₂CH), 63.3 (C₇ₐ), 74.7 (C_{2′}), 116.8 (C_{8′}), 121.2 (C_{6′}), 121.9 (C_{4′a}), 127.2 (C_{5′}), 129.5 (C_{7′}), 154.5 (C_{8′a}), 160.9 (C₃), 174.0 (C₁).

### Example 10

### 2-[3-[[(Chroman-2-yl)methylamino]methyl]benzyl]-1,3-dioxoperhydropyrrolo[1,2-c]imidazole, (j)

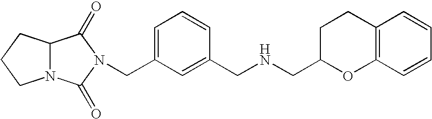

Chromatography: ethyl acetate.

Yield: 40%.

¹H-RMN (CDCl₃, δ): 1.58-2.26 (m, 6H, 2H₆, 2H₇, 2H_{3′}), 2.69-2.96 (m, 4H, CH₂NH, 2H_{4′}), 3.23 (ddd, 1H, J=12.5, 7.6, 5.4 Hz, H₅), 3.69 (dt, 1H, J=11.2, 7.6 Hz, H₅), 3.85 (s, 2H, CH₂Ar), 4.04-4.22 (m, 2H, H₇ₐ, H_{2′}), 4.62 (s, 2H, NCH₂), 6.82 (t, 2H, J=6.8 Hz, H₆, H_{8′}), 7.02-7.11 (m, 2H, H₅, H_{7′}), 7.26-7.34 (m, 4H, ArH).

¹³C-RMN (CDCl₃, δ): 24.7 (C_{3′}), 25.6 (C_{4′}), 27.0 (C₆), 27.5 (C₇), 42.5 (NCH₂), 45.5 (C₅), 53.5, 53.6 (2CH₂NH), 63.4 (C₇ₐ), 75.2 (C₂), 116.8 (C_{8′}), 120.2 (C_{6′}), 122.0 (C_{4′a}), 127.0, 127.2, 127.7, 128.2, 128.8 (C_{7′}, phenyl), 129.5 (C_{5′}), 136.1, 140.7 (phenyl), 154.7 (C_{8′a}), 160.5 (C₃), 173.6 (C₁).

Analysis calculated for O₂₄H₂₇N₃O₃.HCl.3H₂O:

C, 58.12; H, 6.91; N, 8.47

Found: C, 58.19; H, 6.51; N, 8.07

### Example 11

### 2-[4-[[(Chroman-2-yl)methylamino]methyl]benzyl]-1,3-dioxoperhydropyrrolo[1,2-c]imidazole, (k)

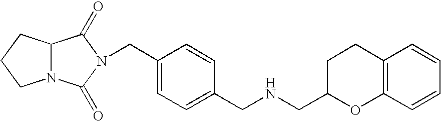

Chromatography: ethyl acetate.

Yield: 44%.

¹H-RMN (CDCl₃, δ): 1.57-2.29 (m, 6H, 2H₆, 2H₇, 2H_{3′}), 2.75-2.95 (m, 4H, CH₂NH, 2H_{4′}), 3.24 (ddd, 1H, J=12.4, 7.3, 5.4 Hz, H₅), 3.69 (dt, 1H, J=11.2, 7.6 Hz, H₅), 3.84 (s, 2H, CH₂Ar), 4.04-4.22 (m, 2H, H₇ₐ, H_{2′}), 4.61 (s, 2H, NCH₂), 6.82 (t, 2H, J=8.1 Hz, H_{6′}, H_{8′}), 7.01-7.11 (m, 2H, H_{5′}, H_{7′}), 7.28-7.38 (m, 4H, ArH).

Analysis calculated for C₂₄H₂₇N₃O₃.HCl.2H₂O:

C, 60.31; H, 6.75; N, 8.79

Found: C, 60.71; H, 6.40; N, 8.52

### Example 12

### (E)-2-[4-[(Chroman-2-yl)methylamino]but-2-enyl]-1,3-dioxoperhydropyrrolo[1,2-c]imidazole, (l)

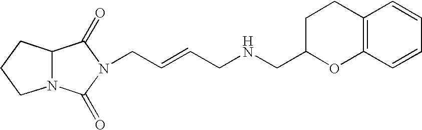

Chromatography: ethyl acetate.

Yield: 43%.

¹H-RMN (CDCl₃, δ): 1.63-2.31 (m, 6H, 2H₆, 2H₇, 2H_{3′}), 2.65-2.93 (m, 4H, CH₂NH, 2H_{4′}), 3.17-3.31 (m, 3H, CH₂NH, H₅), 3.67 (dt, 1H, J=11.2, 7.6 Hz, H₅), 4.03-4.14 (m, 4H, NCH₂, H₇ₐ, H_{2′}), 5.54-5.85 (m, 2H, CH═CH), 6.77-6.85 (m, 2H, H_{6′}, H_{8′}), 7.00-7.10 (m, 2H, H₅, H₇).

¹³C-RMN (CDCl₃, δ): 24.7 (C_{3′}), 25.7 (C_{4′}), 27.1 (C₆), 27.6 (C₇), 40.2 (NCH₂), 45.6 (C₅), 50.9, 53.6 (2CH₂NH), 63.5 (C₇ₐ), 75.1 (C₂), 116.8 (C_{8′}), 120.3 (C_{6′}), 122.1 (C_{4′a}), 124.8 (CH), 127.3 (C_{7′}), 129.6 (C_{5′}), 132.3 (CH), 154.6 (C_{8′a}), 160.4 (C₃), 173.6 (C₁).

Analysis calculated for C₂₀H₂₅N₃O₃.HCl.4H₂O:

C, 51.78; H, 7.39; N, 9.06

Found: C, 52.16; H, 7.00; N, 9.16

### Example 13

### 2-[4-[2-(o-Methoxyphenoxy)ethylamino]butyl]-1,3-dioxoperhydropyrrolo[1,2-c]imidazole (m)

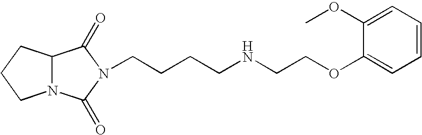

Chromatography: ethyl acetate.

Yield: 38%.

¹H-RMN (CDCl₃, δ): 1.63-1.71 (m, 5H, —(CH₂)₂—, H₇), 1.99-2.29 (m, 3H, 2H₆, H₇), 2.78 (t, 2H, J=6.8 Hz, CH₂NH), 3.01-3.10 (m, 2H, CH₂NH), 3.21 (ddd, 1H, J=11.2, 6.1, 5.6 Hz, H₅), 3.57-3.80 (m, 3H, NCH₂, H₅), 3.83 (s, 3H, OCH₃), 4.00-4.18 (m, 3H, OCH₂, H₇ₐ), 6.87-6.90 (m, 4H, ArH).

¹³C-RMN (CDCl₃, δ): 25.5, 25.6 (—(CH₂)₂—), 26.5, 27.4 (C₆, C₇), 38.4 (NCH₂), 45.4 (C₅), 48.1, 48.6 (2CH₂NH), 63.2 (C₇ₐ), 67.7 (OCH₃), 71.0 (OCH₂), 111.8 (C_{6′}), 120.9 (C_{4′}), 125.9, 129.7 (C_{3′}, C₅), 130.5 (C_{2′}), 147.8 (C_{1′}), 160.6 (C₃), 173.9 (C₁).

Analysis calculated for C₁₉H₂₇N₃O₄.HCl.4H₂O:

C, 48.56; H, 7.72; N, 8.94

Found: C, 48.16; H, 7.32; N, 8.48

### Example 14

### 2-[4-[2-(m-Methoxyphenoxy)ethylamino]butyl]-1,3-dioxoperhydropyrrolo[1,2-c]imidazole, (n)

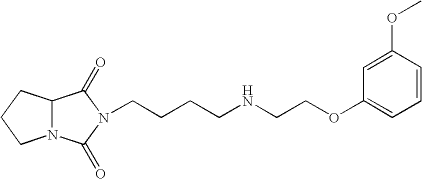

Chromatography: ethyl acetate.

Yield: 38%.

¹H-RMN (CDCl₃, δ): 1.42-1.70 (m, 4H, —(CH₂)₂—), 1.95-2.06 (m, 3H, 2H₆, H₇), 2.10-2.19 (m, 1H, H₇), 2.64 (t, 2H, J=6.8 Hz, CH₂NH), 2.92 (t, 2H, J=5.4 Hz, CH₂NH), 3.16 (ddd, 1H, J=11.2, 7.3, 5.4 Hz, H₅), 3.39 (t, 2H, J=6.3 Hz, NCH₂), 3.59 (dt, 1H, J=11.3, 7.6 Hz, H₅), 3.71 (s, 3H, OCH₃), 3.97-4.07 (m, 3H, OCH₂, H₇ₐ), 6.41-6.47 (m, 3H, H_{2′}, H_{4′}, H_{6′}), 7.10 (t, 1H, J=7.8 Hz, H_{5′}).

¹³C-RMN (CDCl₃, δ): 25.8 (—(CH₂)₂—), 27.0, 27.5 (C₆, C₇), 38.7 (NCH₂), 45.6 (C₅), 48.3, 49.0 (2CH₂NH), 63.2 (C₇ₐ), 67.2, 68.6 (OCH₃, OCH₂), 101.0 (C_{2′}), 106.4, 106.6 (C_{4′}, C_{6′}), 129.8 (C_{5′}), 138.9 (C_{1′}), 160.0 (C_{3′}), 160.8 (C_{3′}), 173.9 (C₁).

Analysis calculated for C₁₉H₂₇N₃O₄. HCl.3H₂O:

C, 50.49; H, 7.58; N, 9.30

Found: C, 50.71; H, 7.18; N, 8.90

### Example 15

### 2-[4-[2-(o-Bromophenoxy)ethylamino]butyl]-1,3-dioxoperhydropyrrolo[1,2-c]imidazole, (o)

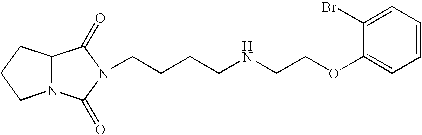

Chromatography: ethyl acetate.

Yield: 48%; m.p. 98-99° C.

¹H-RMN (CDCl₃, δ): 1.46-1.69 (m, 4H, —(CH₂)₂—), 1.98-2.20 (m, 4H, 2H₆, 2H₇), 2.70 (t, 2H, J=6.8 Hz, CH₂NH), 3.00 (t, 2H, J=5.1 Hz, CH₂NH), 3.19 (ddd, 1H, J=11.2, 7.3, 5.4 Hz, H₅), 3.46 (t, 2H, J=7.1 Hz, NCH₂), 3.64 (dt, 1H, J=11.2, 7.6 Hz, H₅), 3.99-4.12 (m, 3H, OCH₂, H₇ₐ), 6.80 (dt, 2H, J=8.1, 8.0 Hz, H_{4′}, H_{6′}), 7.21 (td, 1H, J=5.9, 1.2 Hz, H_{5′}), 7.49 (dd, 1H, J=7.8, 1.4 Hz, H_{3′}).

¹³C-RMN (CDCl₃, δ): 25.6, 26.8 (—(CH₂)₂—), 26.9, 27.4 (C₆, C₇), 38.6 (NCH₂), 45.4 (C₅), 48.2, 48.9 (2CH₂NH), 63.1 (C₇ₐ), 68.4 (OCH₂), 112.2 (C_{2′}), 113.4 (C_{6′}), 121.9 (C_{4′}), 128.3 (C_{5′}), 133.1 (C_{3′}), 155.0 (C_{1′}), 160.6 (C₃), 173.8 (C₁).

Analysis calculated for C₁₈H₂₄BrN₃O₃.HCl.2H₂O:

C, 44.78; H, 6.05; N, 8.70

Found: C, 44.38; H, 5.65; N, 9.05

### Example 16

### 2-[4-[2-(m-Bromophenoxy)ethylamino]butyl]-1,3-dioxoperhydropyrrolo[1,2-c]imidazole, (p)

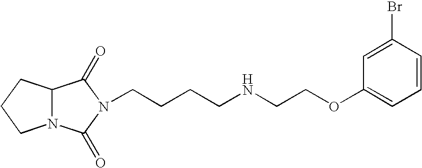

Chromatography: ethyl acetate.

Yield: 42%; m.p. 140-143° C.

¹H-RMN (CDCl₃, δ): 1.42-1.74 (m, 4H, —(CH₂)₂—), 1.94-2.22 (m, 4H, 2H₆, 2H₇), 2.68 (t, 2H, J=7.1 Hz, CH₂NH), 2.96 (t, 2H, J=5.1 Hz, CH₂NH), 3.19 (ddd, 1H, J=11.2, 7.3, 5.1 Hz, H₅), 3.45 (t, 2H, J=7.8 Hz, NCH₂), 3.64 (dt, 1H, J=11.2, 7.6 Hz, H₅), 3.99-4.14 (m, 3H, OCH₂, H₇ₐ), 6.78-6.83 (m, 1H, H_{4′}), 7.02-7.10 (m, 3H, H_{2′}, H_{5′}, H_{6′}).

¹³C-RMN (CDCl₃, δ): 25.6, 26.9, 27.4 (—(CH₂)₂—, C₆, C₇), 38.6 (NCH₂), 45.4 (C₅), 48.4, 49.0 (2CH₂NH), 63.2 (C₇ₐ), 67.3 (OCH₂), 113.4 (C_{6′}), 117.7 (C_{2′}), 122.6 (C_{3′}), 123.8 (C_{4′}), 130.4 (C_{5′}), 159.5 (C_{1′}), 160.7 (C₃), 173.8 (C₁).

Analysis calculated for C₁₈H₂₄BrN₃O₃.HCl.2H₂O:

C, 44.78; H, 6.05; N, 8.70

Found: C, 44.47; H, 5.65; N, 9.30

### Example 17

### 2-[4-[2-(o-Ethylphenoxy)ethylamino]butyl]-1,3-dioxoperhydropyrrolo[1,2-c]imidazole, (q)

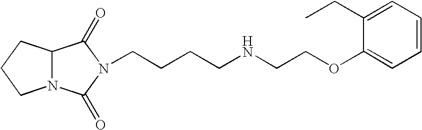

Chromatography: chloroform/methanol, 9.5:0.5.

Yield: 28%; m.p. 115-118° C. (hexane).

¹H-RMN (CDCl₃, δ): 1.19 (t, 3H, J=7.4 Hz, CH₃), 1.59-1.74 (m, 5H, —(CH₂)₂—, H₇), 1.97-2.09 (m, 2H, 2H₆), 2.17-2.28 (m, 1H, H₇), 2.63 (q, 2H, J=7.6 Hz, CH₂CH₃), 2.80 (t, 2H, J=7.1 Hz, CH₂NH), 3.08 (t, 2H, J=5.1 Hz, CH₂NH), 3.17-3.29 (m, 1H, H₅), 3.50 (t, 2H, J=6.8 Hz, NCH₂), 3.61-3.75 (m, 1H, H₅), 4.02-4.15 (m, 3H, OCH₂, H₇ₐ), 6.82-6.94 (m, 2H, H₄, H₆), 7.11-7.17 (m, 2H, H₃, H_{5′}).

¹³C-RMN (CDCl₃, δ): 12.2 (CH₃), 23.2, 25.7, 26.6 (CH₂CH₃, —(CH₂)₂—), 27.0, 27.5 (C₆, C₇), 38.6 (CH₂NCO), 45.5 (C₅), 48.5, 48.9 (2CH₂NH), 63.3 (OCH₂, C₇ₐ), 111.3 (C_{6′}), 120.8 (C_{4′}), 126.8, 129.0 (C_{3′}, C_{5′}), 132.7 (C_{2′}), 156.3 (C_{1′}), 160.8 (C₃), 167.4 (C₁).

Analysis calculated for C₂H₂₉N₃O₃.HCl.2H₂O:

C, 55.61; H, 7.93; N, 9.73

Found: C, 55.89; H, 7.53; N, 9.81

### Example 18

### 2-[4-[2-(m-Ethylphenoxy)ethylamino]butyl]-1,3-dioxoperhydropyrrolo[1,2-c]imidazole, (r)

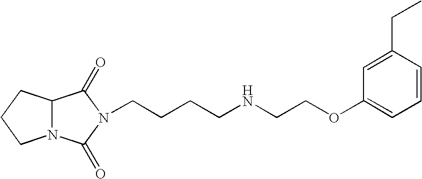

Chromatography: ethyl acetate.

Yield: 43%.

¹H-RMN (CDCl₃, δ): 1.24 (t, 3H, J=7.6 Hz, CH₃), 1.59-1.74 (m, 5H, —(CH₂)₂—, H₇), 1.97-2.09 (m, 2H, 2H₆), 2.17-2.28 (m, 1H, H₇), 2.63-2.74 (m, 4H, CH₂CH₃, CH₂NH), 3.12 (t, 2H, J=5.1 Hz, CH₂NH), 3.17-3.29 (m, 1H, H₅), 3.50 (t, 2H, J=7.1 Hz, NCH₂), 3.61-3.75 (m, 1H, H₅), 4.02-4.15 (m, 3H, OCH₂, H₇ₐ), 6.72-6.86 (m, 3H, H_{2′}, H_{4′}, H_{6′}), 7.21 (t, 1H, J=7.8 Hz, H_{5′}).

¹³C-RMN (CDCl₃, δ): 15.5 (CH₃), 25.5, 25.8, 27.0 (CH₂CH₃, —(CH₂)₂—), 27.3, 27.6 (C₆, C₇), 38.8 (NCH₂); 45.6 (C₅), 48.7, 49.0 (2CH₂NH), 63.4 (C₇ₐ), 66.9 (OCH₂), 111.5 (C_{2′}), 114.4 (C_{6′}), 120.6 (C_{4′}), 129.3 (C_{5′}), 146.0 (C_{3′}), 158.9 (C_{1′}), 160.9 (C₃), 174.0 (C₁).

Analysis calculated for C₂₀H₂₉N₃O₃.HCl.H₂O:

C, 58.03; H, 7.79; N, 10.15

Found: C, 57.92; H, 7.91; N, 10.12

### Example 19

### 2-[4-[2-(o-Isopropylphenoxy)ethylamino]butyl]-1,3-dioxoperhydropyrrolo[1,2-c]imidazole, (s)

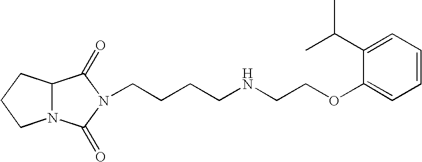

Chromatography: ethyl acetate.

Yield: 23%.

¹H-RMN (CDCl₃, δ): 1.21 (d, 6H, J=7.9 Hz, 2CH₃), 1.44-1.76 (m, 5H, —(CH₂)₂—, H₇), 1.95-2.32 (m, 3H, 2H₆, H₇), 2.71 (t, 2H, J=6.8 Hz, CH₂NH), 3.02 (t, 2H, J=5.1 Hz, CH₂NH), 3.17-3.37 (m, 2H, CH, H₅), 3.49 (t, 2H, J=7.1 Hz, NCH₂), 3.67 (dt, 1H, J=7.6, 3.9 Hz, H₅), 4.00-4.09 (m, 3H, OCH₂, H₇ₐ), 6.82-6.96 (m, 2H, H_{4′}, H_{6′}), 7.09-7.22 (m, 2H, H_{3′}, H_{5′}).

¹³C-RMN (CDCl₃, δ): 22.7 (CH₃), 25.9, 26.9, 27.0, 27.3, 27.6 (—(CH₂)₂—, CH, C₆, C₇), 38.8 (NCH₂), 45.6 (C₅), 49.0, 49.2 (2CH₂NH), 63.4 (C₇ₐ), 67.5 (OCH₂), 111.5 (C_{6′}), 120.8 (C_{4′}), 126.1, 126.6 (C_{3′}, C_{5′}), 135.3 (C_{2′}), 157.5 (C_{1′}), 160.8 (C₃), 173.9 (C₁).

### Example 20

### 2-[4-[(2-Quinolyl)methylamino]butyl]-1,3-dioxoperhydropyrrolo[1,2-c]imidazole, (t)

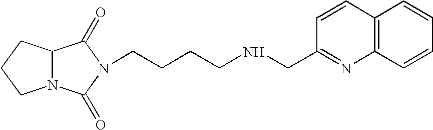

Chromatography: ethyl acetate.

Yield: 33%; m.p. 125-126° C.

IR (CHCl₃, cm⁻¹): 1770, 1708 (CONCON), 1601, 1504, 1442, 1416 (Ar).

¹H-RMN (CDCl₃, δ): 1.52-1.67 (m, 5H, —(CH₂)₂—, H₇), 1.90-2.27 (m, 3H, 2H₆, H₇), 2.50 (t, 2H, J=6.3 Hz, CH₂NH), 3.01-3.24 (m, 1H, H₅), 3.42 (t, 2H, J=6.8 Hz, NCH₂), 3.53-3.69 (m, 1H, H₅), 3.91-4.00 (m, 3H, CH₂Ar, H₇ₐ), 7.47 (t, J=7.1 Hz, 1H, H_{6′}), 7.62-7.77 (m, 3H, H_{3′}, H_{5′}, H_{7′}), 8.02 (d, J=8.3 Hz, 1H, H_{4′}), 8.11 (d, J=8.5 Hz, 1H, H_{8′}).

¹³C-RMN (CDCl₃, δ): 24.4, 25.8, 26.8, 27.4 (2CH₂, C₆, C₇), 38.7 (NCH₂), 45.4 (C₅), 53.8, 54.1 (CH₂Ar, CH₂NH), 63.1 (C₇ₐ), 120.9 (C_{3′}), 126.0 (C_{6′}), 127.2, 127.4 (C_{5′}, C_{8′}), 128.9, 129.2 (C_{4′}, C_{7′}), 130.7 (C_{4′a}), 155.9 (C_{2′}), 160.5 (C_{8′a}), 160.7 (C₃), 173.8 (C₁).

### Example 21

### 2-[4-[2-(o-Ethoxyphenoxy)ethylamino]butyl]-1,3-dioxoperhydropyrrolo[1,2-c]imidazole, (u)

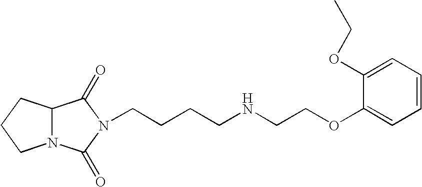

Chromatography: ethyl acetate.

Yield: 30%.

¹H-RMN (CDCl₃, δ): 1.43 (t, 3H, J=6.8 Hz, CH₃), 1.62-1.72 (m, 5H, —(CH₂)₂—, H₇), 1.94-2.27 (m, 3H, 2H₆, H₇), 2.78 (t, 2H, J=6.6 Hz, CH₂NH), 3.06 (t, 2H, J=5.1 Hz, CH₂NH), 3.22 (ddd, 1H, J=12.4, 7.3, 5.1 Hz, H₅), 3.44-3.72 (m, 3H, NCH₂, H₅), 4.01-4.17 (m, 4H, OCH₂, H₇ₐ, CH₂CH₃), 6.87-6.92 (m, 4H, ArH).

¹³C-RMN (CDCl₃, δ): 14.8 (CH₃), 25.6, 26.4, 26.8, 27.4 (—(CH₂)₂—, C₆, C₇), 38.5 (NCH₂), 45.4 (C₅), 48.3, 48.7 (2CH₂NH), 63.2 (C₇ₐ), 64.3 (CH₂CH₃), 68.3 (OCH₂), 113.6, 115.1, 120.9, 121.8 (C_{6′}, C_{4′}, C_{3′}, C₅), 148.3 (C_{2′}), 149.1 (C_{1′}), 160.7 (C₃), 173.8 (C₁).

### Example 22

### 2-[4-[2-(o-Isopropoxyphenoxy)ethylamino]butyl]-1,3-dioxoperhydropyrrolo[1,2-c]imidazole, (v)

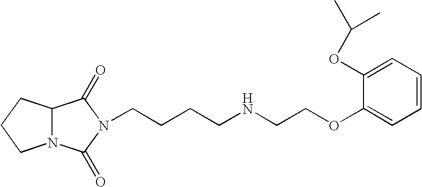

Chromatography: ethyl acetate.

Yield: 23%.

¹H-RMN (CDCl₃, δ): 1.33 (d, 6H, J=6.1 Hz, 2CH₃), 1.55-1.71 (m, 4H, —(CH₂)₂—), 2.04-2.26 (m, 4H, 2H₆, 2H₇), 2.72 (q, 2H, J=6.2 Hz, CH₂NH), 3.02 (q, 2H, J=5.1 Hz, CH₂NH), 3.23 (ddd, 1H, J=12.5, 7.3, 5.1 Hz, H₅), 3.48 (t, 2H, J=6.8 Hz, NCH₂), 3.67 (dt, 1H, J=11.0, 7.8 Hz, H₅), 4.02-4.13 (m, 3H, OCH₂, H₇ₐ), 4.45 (sept, 1H, J=6.1 Hz, CH), 6.89-6.92 (m, 4H, ArH).

¹³C-RMN (CDCl₃, δ): 22.1, 22.2 (CH₃), 25.8, 27.0, 27.5 (—(CH₂)₂—, C₆, C₇), 38.7 (NCH₂), 45.5 (C₅), 48.7, 49.0 (2CH₂NH), 63.3 (C₇ₐ), 68.7 (OCH₂), 72.1 (OCH), 115.5, 116.7, 117.6, 121.8 (C_{6′}, C_{4′}, C_{3′}, C_{5′}), 145.5 (C_{2′}), 151.2 (C_{1′}), 160.3 (C₃), 173.9 (C₁).

Analysis calculated for C₂₀H₂₉N₃O₃.HCl.H₂:

C, 58.03; H, 7.79; N, 10.15

Found: C, 57.92; H, 7.91; N, 10.12

### Example 23

### 2-[4-[2-[m-(Trifluoromethyl)phenoxy]ethylamino]butyl]-1,3-dioxoperhydropyrrolo[1,2-c]imidazole, (w)

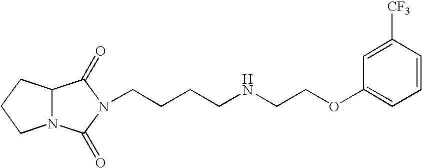

Chromatography: ethyl acetate/ethanol, 9:1.

Yield: 30%.

¹H-RMN (CDCl₃, δ): 1.50-1.72 (m, 5H, —(CH₂)₂—, H₇), 1.91-2.21 (m, 3H, 2H₆, H₇), 2.72 (t, 2H, J=6.8 Hz, CH₂NH), 3.01 (t, 2H, J=5.2 Hz, CH₂NH), 3.11-3.22 (m, 1H, H₅), 3.42 (t, 2H, J=6.8 Hz, NCH₂), 3.58-3.67 (m, 1H, H₅), 3.96-4.29 (m, 3H, OCH₂, H₇ₐ), 7.05-7.17 (m, 3H, H₂, H₄, H₆), 7.32 (t, 1H, J=7.9 Hz, H₅).

Analysis calculated for C₁₉H₂₄F₃N₃O₃.HCl.H₂O:

C, 50.28; H, 6.00; N, 9.26

Found: C, 50.62; H, 6.10; N, 8.75

### Example 24

### 2-[4-[2-(1,1′-Biphenyl-2-yloxy)ethylamino]butyl]-1,3-dioxoperhydropyrrolo[1,2-c]imidazole, (x)

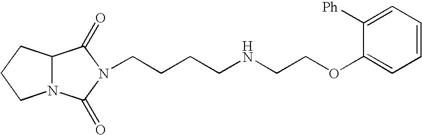

Chromatography: ethyl acetate/ethanol, 9:1.

Yield: 35%.

¹H-RMN (CDCl₃, δ): 1.22-1.77 (m, 4H, —(CH₂)₂—)), 1.93-2.08 (m, 3H, 2H₆, H₇), 2.16-2.30 (m, 1H, H₇), 2.57 (t, 2H, J=7.0 Hz, CH₂NH), 2.95 (t, 2H, J=5.1 Hz, CH₂NH), 3.12-3.21 (m, 1H, H₅), 3.37 (t, 2H, J=7.0 Hz, NCH₂), 3.53-3.62 (m, 1H, H₅), 3.96-4.05 (m, 3H, OCH₂, H₇ₐ), 6.79-7.00 (m, 2H, ArH), 7.18-7.47 (m, 7H, ArH).

¹³C-RMN (CDCl₃, δ): 25.7, 26.9, 27.0, 27.5 (—(CH₂)₂—, C₆, C₇), 38.7 (NCH₂), 45.5 (C₅), 48.5, 48.9 (2CH₂NH), 63.3 (C₇ₐ), 68.1 (OCH₂), 113.3 (C_{6′}), 121.3 (C_{4′}), 126.9, 128.6 (C_{3′}, C_{5′}), 127.9, 129.5, 130.8 (5CH-Ph), 131.4 (C_{2′}), 138.5 (C-Ph), 155.7 (C_{1′}), 160.8 (C₃), 173.9 (C₁).

Analysis calculated for C₂₄H₂₉N₃O₃. HCl.6H₂O:

C, 52.21; H, 7.67; N, 7.61

Found: C, 52.61; H, 7.27; N, 8.01

### Example 25

### 2-[4-[2-[o-(Acetylamino)phenoxy]ethylamino]butyl]-1,3-dioxoperhydropyrrolo[1,2-c]imidazole, (y)

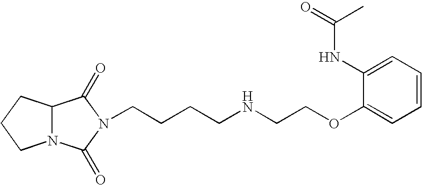

Chromatography: ethyl acetate/ethanol, 9:1.

Yield: 24%

¹H-RMN (CDCl₃, δ): 1.60-1.71 (m, 5H, —(CH₂)₂—, H₇), 1.96-2.17 (m, 3H, 2H₆, H₇), 2.21 (s, 3H, CH₃), 2.78 (t, 2H, J=6.9 Hz, CH₂NH), 3.09 (t, 2H, J=4.9 Hz, CH₂NH), 3.14-3.26 (m, 1H, H₅), 3.43 (t, 2H, J=7.1 Hz, NCH₂), 3.62 (dt, 1H, J=11.2, 7.8 Hz, H₅), 3.93-4.19 (m, 3H, OCH₂, H₇ₐ), 6.82-6.99 (m, 3H, ArH), 8.14 (dd, 1H, J=7.3, 1.7 Hz, ArH), 8.68 (br s, 1H, NH).

¹³C-RMN (CDCl₃, δ): 24.6 (CH₃), 25.2, 25.3, 26.9, 27.4 (—(CH₂)₂—, C₆, C₇), 38.0 (NCH₂), 45.3 (C₅), 47.7, 48.2 (2CH₂NH), 63.2 (C₇ₐ), 66.8 (OCH₂), 112.2 (C_{6′}), 121.5, 121.7 (C_{3′}, C_{4′}), 124.0 (C_{5′}), 128.1 (C_{2′}), 147.4 (C_{1′}), 160.6 (C₃), 169.0 (CONH), 173.9 (C₁).

Analysis calculated for C₂₀H₈N₄O₄.HCl.3H₂O:

C, 50.15; H, 7.37; N, 11.70

Found: C, 50.55; H, 7.75; N, 11.98

### Example 26

### 2-[4-[2-[m-(Acetylamino)phenoxy]ethylamino]butyl]-1,3-dioxoperhydropyrrolo[1,2-c]imidazole, (z)

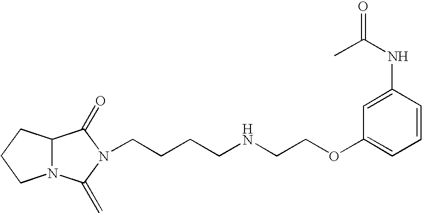

Chromatography: ethyl acetate/methanol, 9:1.

Yield: 31%

¹H-RMN (CDCl₃, δ): 1.60-1.72 (m, 5H, —(CH₂)₂—, H₇), 1.96-2.15 (m, 3H, 2H₆, H₇), 2.21 (s, 3H, CH₃), 2.82 (t, 2H, J=6.1 Hz, CH₂NH), 3.08 (t, 2H, J=5.8 Hz, CH₂NH), 3.19 (ddd, 1H, J=11.2, 7.6, 5.1 Hz, H₅), 3.44 (t, 2H, J=6.9 Hz, NCH₂), 3.62 (dt, 1H, J=11.2, 7.8 Hz, H₅), 4.00-4.10 (m, 3H, OCH₂, H₇ₐ), 4.54 (br s, 1H, NH), 6.54-6.57 (m, 1H, ArH), 6.97-7.19 (m, 3H, ArH).

¹³C-RMN (CDCl₃): 24.7 (CH₃), 25.4, 25.9, 27.2, 27.6 (—(CH₂)₂—C₆, C₇), 38.5 (NCH₂), 45.6 (C₅), 48.6, 48.8 (2CH₂NH), 63.5 (C₇ₐ), 65.6 (OCH₂), 106.8 (C_{2′}), 110.2 (C_{6′}), 113.1 (C_{4′}), 129.9 (C_{5′}), 138.6 (C_{3′}), 158.7 (C_{1′}), 160.9 (C₃), 169.4 (CONH), 174.2 (C₁).

Analysis calculated for C₂₀H₂₈N₄O₄.HCl.3H₂O:

C, 50.15; H, 7.37; N, 11.70

Found: C, 50.65; H, 7.65; N, 12.03

### Example 27

### 2-[4-[2-[o-(Ethoxycarbonyl)phenoxy]ethylamino]butyl]-1,3-dioxoperhydropyrrolo[1,2-c]imidazole, (aa)

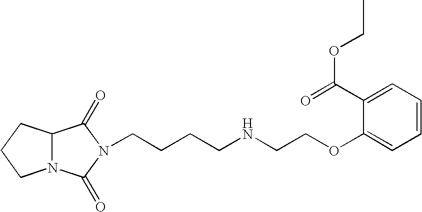

Chromatography: ethyl acetate/ethanol, 9:1.

Yield: 25%

¹H-RMN (CDCl₃, δ): 1.39 (t, J=7.2 Hz, CH₃CH₂), 1.66-1.83 (m, 5H, —(CH₂)₂—, H₇), 1.94-2.45 (m, 5H, 2H₆, H₇, CH₂NH), 3.15-3.27 (m, 3H, CH₂NH, H₅), 3.44-3.54 (m, 2H, NCH₂), 3.66 (dt, 1H, J=11.2, 7.6 Hz, H₅), 4.07 (dd, 1H, J=9.0, 7.3 Hz, H₇ₐ), 4.33 (q, 2H, J=7.1 Hz, CH₃CH₂), 4.54 (t, 2H, J=4.6 Hz, OCH₂), 7.01-7.11 (m, 2H, ArH), 7.50 (td, 1H, J=7.8, 1.7 Hz, ArH), 7.84 (dd, 1H, J=7.8, 1.5 Hz, ArH).

¹³C-RMN (CDCl₃, δ): 14.0 (CH₃CH₂), 22.9, 24.8, 26.9, 27.4 (—(CH₂)₂—, C₆, C₇), 37.5 (NCH₂), 45.3 (C₅), 47.3, 47.4 (2CH₂NH), 61.5 (CH₃CH₂), 63.2 (C₇ₐ), 65.7 (OCH₂), 116.2 (C_{6′}), 120.2 (C_{2′}), 122.3 (C_{4′}), 131.6 (C_{3′}), 134.4 (C_{5′}), 158.0 (C_{1′}), 160.4 (C₃), 166.6 (COO), 173.8 (C₁).

Analysis calculated for C₃HCl.3H₂O:

C, 51.06; H, 7.35; N, 8.51

Found: C, 51.36; H, 7.42; N, 8.68

### Example 28

### 2-[4-[2-(5,6,7,8-Tetrahydronaphth-1-yloxy)ethylamino]butyl]-1,3-dioxoperhydropyrrolo[1,2-c]imidazole, (bb)

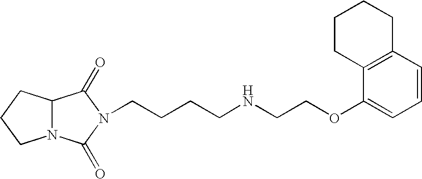

Chromatography: ethyl acetate/ethanol, 9:1.

Yield: 32%

¹H-RMN (CDCl₃, δ): 1.52-1.77 (m, 9H, —(CH₂)₄—, H₇), 1.98-2.15 (m, 2H, 2H₆), 2.17-2.29 (m, 1H, H₇), 2.64-2.78 (m, 6H, CH₂NH, 2CH₂Ar), 3.01 (t, J=5.1 Hz, CH₂NH), 3.17-3.29 (m, 1H, H₅), 3.49 (t, 2H, J=7.1 Hz, NCH₂), 3.65 (dt, 1H, J=11.2, 7.6 Hz, H₅), 4.02-4.10 (m, 3H, OCH₂, H₇ₐ), 6.57 (d, 1H, J=8.1 Hz, ArH), 6.62 (d, 1H, J=7.6 Hz, ArH), 7.03 (t, 1H, J=7.8 Hz, ArH).

¹³C-RMN (CDCl₃, δ): 22.8, 23.1, 25.8, 27.0, 27.2, 27.6 (—(CH₂)₂—, —(CH₂)₄—, C₆, C₇), 38.8 (NCH₂), 45.5 (C₅), 48.8, 49.1 (2CH₂NH), 63.3 (C₇ₐ), 67.1 (OCH₂), 107.9 (C_{2′}), 121.6 (C_{4′}), 125.6 (C_{3′}), 126.1 (C_{8′a}), 138.6 (C_{4′a}), 156.5 (C_{1′}), 160.8 (C₃), 173.9 (C₁).

Analysis calculated for C₂₂H₃₁N₃O₃.HCl.3H₂O:

C, 55.51; H, 8.05; N, 8.83

Found: C, 55.18; H, 7.77; N, 8.90

### Example 29

### 2-[4-[2-(2,3-Dimethylphenoxy)ethylamino]butyl]-1,3-dioxoperhydropyrrolo[1,2-c]imidazole, (cc)

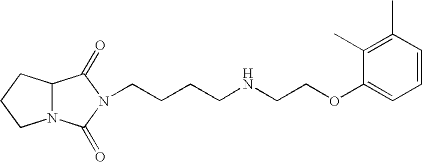

Chromatography: ethyl acetate/methanol, 9:1.

Yield: 30%

¹H-RMN (CDCl₃, δ): 1.53-1.73 (m, 5H, —(CH₂)₂—, H₇), 1.98-2.23 (m, 3H, 2H₆, H₇), 2.14 (s, 3H, CH₃), 2.27 (s, 3H, CH₃), 2.72 (t, 2H, J=6.8 Hz, CH₂NH), 3.02 (t, 2H, J=4.9 Hz, CH₂NH), 3.17-3.29 (m, 1H, H₅), 3.49 (t, 2H, J=6.8 Hz, NCH₂), 3.67 (dt, 1H, J=11.2, 7.6 Hz, H₅), 4.02-4.10 (m, 3H, OCH₂, H₇ₐ), 6.70 (d, 1H, J=8.3 Hz, ArH), 6.77 (d, 1H, J=7.6 Hz, ArH), 7.03 (t, 1H, J=7.8 Hz, ArH).

¹³C-RMN (CDCl₃, δ): 11.7, 20.1 (2CH₃), 25.9, 27.0, 27.2, 27.6 (—(CH₂)₂—, C₆, C₇), 38.8 (NCH₂), 45.6 (C₅), 49.0, 49.2 (2CH₂NH), 63.4 (C₇ₐ), 67.7 (OCH₂), 109.2 (C_{6′}), 122.4 (C_{4′}), 125.8 (C_{5′}), 137.9, 138.1 (C_{2′}, C₃), 155.9 (C_{1′}), 160.5 (C₃), 173.9 (C₁).

Analysis calculated for C₂₀H₂₉N₃O₃.HCl.3H₂O:

C, 53.38; H, 8.06; N, 9.34

Found: C, 52.99; H, 8.15; N, 9.74

### Example 30

### 2-[4-[(Chroman-2-yl)methylamino]butyl]-1,4-dioxoperhydropyrido[1,2-a]pyrazine, (dd)

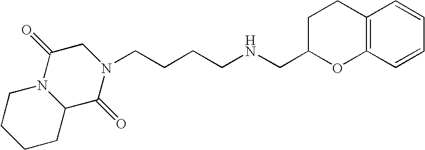

Chromatography: ethyl acetate.

Yield: 35%.

¹H-RMN (CDCl₃): 1.40-1.68 (m, 8H, —(CH₂)₂—, 2H₇, H₈ₐₓ, H₉ₐₓ), 1.96-2.07 (m, 3H, H_{8ec}, 2H_{3′}), 2.33-2.58 (m, 2H, H_{9ec}, H₆ₐₓ), 2.70-2.96 (m, 6H, 2NHCH₂, 2H_{4′}), 3.41 (t, 2H, J=6.6 Hz, NCH₂), 3.82 (d, 2H, J=11.7 Hz, H₉ₐ), 3.96 (s, 2H, 2H₃), 4.14-4.19 (m, 1H, H_{2′}), 4.67 (d, 1H, J=12.9 Hz, H_{6ec}), 6.83 (t, 2H, J=7.6 Hz, H_{6′}, H_{8′}), 7.02-7.11 (m, 2H, H_{5′}, H_{7′}).

¹³C-RMN (CDCl₃, δ): 24.2, 24.4, 24.6, 25.6, 26.9 (—(CH₂)₂—, C₇, C₈, C_{3′}, C_{4′}), 31.3 (C₉), 42.4 (C₆), 45.7 (NCH₂), 49.2, 49.3 (NHCH₂, C₃), 54.0 (NHCH₂), 59.2 (C₉ₐ), 74.8 (C_{2′}), 116.8 (C_{8′}), 120.2 (C_{6′}), 122.0 (C_{4a′}), 127.2 (C_{7′}), 129.5 (C_{5′}), 154.4 (C_{8a′}), 161.3 (C₄), 164.9 (C₁).

Analysis calculated for C₂₂H₃₁N₃O₃.HCl.H₂O:

C, 60.06; H, 7.79; N, 9.55

Found: C, 60.23; H, 7.43; N, 9.22

### Example 31

### (Z)-2-[4-[(Chroman-2-yl)methylamino]but-2-enyl]-1,4-dioxoperhydropyrrolo[1,2-c]imidazole, (ee)

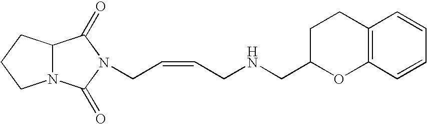

Chromatography: ethyl acetate.

Yield: 38%.

¹H-RMN (CDCl₃, δ): 1.59-2.32 (m, 6H, 2H₆, 2H₇, 2H_{3′}), 2.70-2.86 (m, 4H, CH₂NH, 2H_{4′}), 3.24 (ddd, 1H, J=11.2, 7.6, 5.4 Hz, H₅), 3.50 (d, 2H, J=6.6 Hz, CH₂NH), 3.68 (dt, 1H, J=11.2, 7.8 Hz, H₅), 4.03-4.19 (m, 4H, NCH₂, H₇ₐ, H_{2′}), 5.47-5.57 (m, 1H, CH), 5.70-5.82 (m, 1H, CH), 6.79-6.86 (m, 2H, H₆, H_{8′}), 7.02-7.07 (m, 2H, H₅, H₇).

¹³C-RMN (CDCl₃, δ): 24.5, 25.4, 26.8, 27.3 (C_{3′}, C_{4′}, C₆, C₇), 35.7 (NCH₂), 45.3, 45.9 (C₅, CH₂NH), 53.6 (CH₂NH), 63.2 (C₇ₐ), 75.0 (C₂), 116.6 (C_{8′}), 119.9 (C_{6′}), 121.8 (C_{4′a}), 124.3 (CH), 127.0 (C_{7′}), 129.3 (C_{5′}), 132.7 (CH), 155.8 (C_{8′a}), 156.2 (C₃), 172.4 (C₁).

Analysis calculated for C₂₀H₂₅N₃O₃.HCl.4H₂:

C, 51.78; H, 7.39; N, 9.06

Found: C, 51.42; H, 7.02; N, 8.75

### Example 32

### 3-[4-[2-(o-Ethoxyphenoxy)ethylamino]butyl]-2,4-dioxothiazolidine, (ff)

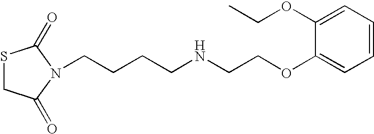

Chromatography: ethyl acetate/ethanol, 9:1.

Yield: 55%; m.p. 70-74° C.

¹H-RMN (CDCl₃, δ): 1.44 (t, 3H, J=7.0 Hz, CH₃), 1.57-1.74 (m, 4H, —(CH₂)₂—), 2.83 (t, 2H, J=7.0 Hz, CH₂NH), 3.13 (t, 2H, J=5.0 Hz, CH₂NH), 3.29 (t, 2H, J=7.5 Hz, NCH₂), 3.96 (s, 2H, 2H₅), 4.08 (q, 2H, J=7.0 Hz, CH₂CH₃), 4.19 (t, 2H, J=5.0 Hz, OCH₂), 6.86-6.97 (m, 4H, ArH).

¹³C-RMN (CDCl₃, δ): 14.9 (CH₃), 24.0, 25.2 (—(CH₂)₂—), 33.8 (C₅), 41.6 (NCH₂), 48.2, 48.5 (2CH₂NH), 64.4 (CH₂CH₃), 68.3 (OCH₂), 113.6, 115.1, 121.0, 121.8 (C_{6′}, C_{4′}, C_{3′}, C_{5′}), 148.3, 149.1 (C_{1′}, C_{2′}), 171.3 (C₂, C₄).

Analysis calculated for C₁₇H₂₄N₂O₄S.HCl.½H₂O:

C, 51.31; H, 6.59; N, 7.04

Found: C, 51.36; H, 7.04; N, 6.66

### Example 33

### 3-[6-[2-(o-Ethoxyphenoxy)ethylamino]hexyl]-2,4-dioxothiazolidine, (gg)

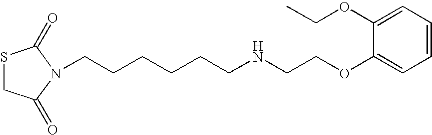

Chromatography: ethyl acetate/ethanol, 9:1.

Yield: 48%; m.p. 92-94° C.

¹H-RMN (CDCl₃, δ): 1.18-1.66 (m, 11H, —(CH₂)₄—, CH₃), 2.73 (t, 2H, J=7.1 Hz, CH₂NH), 3.06 (t, 2H, J=5.3 Hz, CH₂NH), 3.46 (br s, 1H, NH), 3.60 (t, 2H, J=7.4 Hz, NCH₂), 3.92 (s, 2H, 2H₅), 4.06 (q, 2H, J=7.0 Hz, CH₂CH₃), 4.15 (t, 2H, J=5.0 Hz, OCH₂), 6.81-6.97 (m, 4H, ArH).

¹³C-RMN (CDCl₃, δ): 14.8 (CH₃), 26.3, 26.6, 27.3, 29.1 (—(CH₂)₄—), 33.6 (C₅), 41.9 (NCH₂), 48.4, 49.2 (2CH₂NH), 64.4 (CH₂CH₃), 68.3 (OCH₂), 113.6, 115.1, 121.0, 121.8 (C_{6′}, C_{4′}, C_{3′}, C_{5′}), 148.3, 149.1 (C_{1′}, C_{2′}), 171.3 (C₂, C₄).

Analysis calculated for C₁₇H₂₄N₂O₄S.HCl.H₂O:

C, 52.46; H, 7.18; N, 6.44

Found: C, 52.64; H, 6.99; N, 6.45

### Example 34

### 3-[8-[2-(o-Ethoxyphenoxy)ethylamino]octyl]-2,4-dioxothiazolidine, (hh)

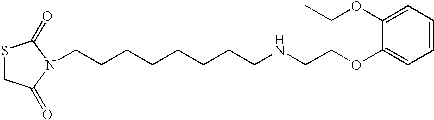

Chromatography: ethyl acetate/ethanol, 9:1.

Yield: 48%; m.p. 105-108° C.

¹H-RMN (CDCl₃, δ): 1.07-1.54 (m, 15H, —(CH₂)₆—, CH₃), 2.73 (t, 2H, J=7.4 Hz, CH₂NH), 3.04 (t, 2H, J=5.2 Hz, CH₂NH), 3.44 (br s, 1H, NH), 3.54 (t, 2H, J=7.4 Hz, NCH₂), 3.87 (s, 2H, 2H₅), 4.01 (q, 2H, J=7.0 Hz, CH₂CH₃), 4.13 (t, 2H, J=5.2 Hz, OCH₂), 6.76-6.92 (m, 4H, ArH).

¹³C-RMN (CDCl₃, δ): 14.7 (CH₃), 26.4, 26.8, 27.3, 28.7, 28.8, 29.0 (—(CH₂)₆—), 33.5 (C₅), 41.9 (NCH₂), 48.1, 49.1 (2CH₂NH), 64.3 (CH₂CH₃), 68.0 (OCH₂), 113.5, 115.1, 120.9, 121.9 (C_{6′}, C_{4′}, C_{3′}, C_{5′}), 148.3, 149.2 (C_{1′}, C_{2′}), 171.3 (C₂, C₄).

Analysis calculated for C₂₁H₃₂N₂O₄S.HCl.½H₂O:

C, 55.55; H, 7.55; N, 6.17

Found: C, 55.78; H, 7.34; N, 6.04

### Example 35

### 2-[4-[2-(o-Ethoxyphenoxy)ethylamino]butyl]-1,3-dioxoperhydroimidazo[1,5-a]pyridine, (ii)

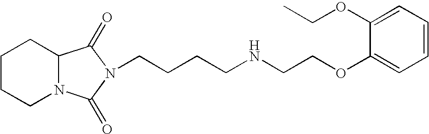

Chromatography: ethyl acetate/ethanol, 7:3.

Yield: 31%.

¹H-RMN (CDCl₃, δ): 1.28-1.74 (m, 11H, —(CH₂)₂—, CH₃, H₆ₐₓ, H₇ₐₓ, H₈ₐₓ, H_{6ec}), 1.94-2.03 (m, 1H, H_{7ec}), 2.14-2.21 (m, 1H, H_{8ec}), 2.73-2.87 (m, 3H, H₅ₐₓ, CH₂NH), 3.11 (t, 2H, J=5.2 Hz, CH₂NH), 3.51 (t, 2H, J=6.5 Hz, NCH₂), 3.69-3.79 (m, 1H, H₈ₐ), 4.00-4.12 (m, 3H, CH₂CH₃, H_{5ec}), 4.18 (t, 2H, J=5.1 Hz, OCH₂), 6.77-6.87 (m, 4H, ArH).

¹³C-RMN (CDCl₃, δ): 15.1 (CH₃), 22.9, 25.1, 26.0, 27.9, 29.8 (—(CH₂)₂—, C₆, C₇, C₈), 38.3 (NCH₂), 39.4 (C₅), 48.3, 48.8 (2CH₂NH), 57.5 (C₈ₐ), 64.6, 68.1 (CH₂CH₃, OCH₂), 113.7, 115.4, 121.2, 122.2 (C_{6′}, C_{4′}, C_{3′}, C_{5′}), 148.3, 149.3 (C_{1′}, C_{2′}), 154.1 (C₃), 173.4 (C₁).

Analysis calculated for C₂₁H₃₁N₃O₄.HCl.H₂O:

C, 56.81; H, 7.72; N, 9.46

Found: C, 57.38; H, 8.00; N, 9.02

### Example 36

### 2-[6-[2-(o-Ethoxyphenoxy)ethylamino]hexyl]-1,3-dioxoperhydroimidazo[1,5-a]pyridine, (jj)

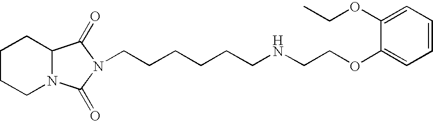

Chromatography: ethyl acetate/ethanol, 8:2.

Yield: 49%.

¹H-RMN (CDCl₃, 5): 1.23-1.63 (m, 14H, —(CH₂)₄—, CH₃, H₆ₐₓ, H₇ₐₓ, H₈ₐₓ), 1.71-1.75 (m, 1H, H_{6ec}), 1.96-2.01 (m, 1H, H_{7ec}), 2.18-2.22 (m, 1H, H_{8ec}), 2.70 (t, 2H, J=7.3 Hz, CH₂NH), 2.77-2.87 (m, 1H, H₅ₐₓ), 3.03 (t, 2H, J=5.3 Hz, CH₂NH), 3.48 (t, 2H, J=7.3 Hz, NCH₂), 3.73 (dd, 1H, J=11.9, 4.1 Hz, H₈ₐ), 4.07 (q, 2H, J=7.0 Hz, CH₂CH₃), 4.14 (t, 2H, J=5.3 Hz, OCH₂), 4.18-4.19 (m, 1H, H_{5ec}), 6.86-7.26 (m, 4H, ArH).

¹³C-RMN (CDCl₃, δ): 14.7 (CH₃), 22.6, 24.8, 26.3, 26.6, 27.6, 27.0, 29.3 (—(CH₂)₄—, C₆, C₇, C₈), 38.3 (NCH₂), 39.1 (C₅), 48.4, 49.3 (2CH₂NH), 57.1 (C₈ₐ), 64.3, 68.4 (CH₂CH₃, OCH₂), 113.5, 114.9, 120.9, 121.6 (C_{6′}, C_{4′}, C_{3′}, C_{5′}), 148.3, 148.7 (C_{1′}, C_{2′}), 154.4 (C₃), 173.1 (C₁).

Analysis calculated for C₂₃H₃₅N₃O₄.HCl.7/2H₂O:

C, 53.43; H, 8.38; N, 8.13

Found: C, 53.18; H, 7.82; N, 7.60

### Example 37

### 2-[4-[(2-Quinolyl)methylamino]butyl]-1,3-dioxoperhydroimidazo[1,5-a]pyridine, (kk)

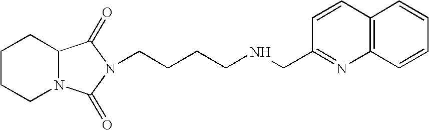

Chromatography: ethyl acetate/ethanol, 7:3.

Yield: 45%; m.p. 206-208° C.

¹H-RMN (CDCl_{3′}): 1.21-1.77 (m, 8H, —(CH₂)₂—, H₆ₐₓ, H₇ₐₓ, H₈ₐₓ, H_{6ec}), 1.95-1.99 (m, 1H, H_{7ec}), 2.16-2.22 (m, 1H, H_{8ec}), 2.76-2.86 (m, 3H, H₅ₐₓ, CH₂NH), 3.24 (br s, 1H, NH), 3.53 (t, 2H, J=6.9 Hz, NCH₂), 3.73 (dd, 1H, J=12.1, 4.4 Hz, H₈ₐ), 4.12-4.18 (m, 3H, CH₂Ar, H_{5ec}), 7.46 (d, 1H, J=8.4 Hz, H₃), 7.44-7.54 (m, 1H, H_{6′}), 7.67-7.73 (m, 1H, H_{7′}), 7.81 (dd, 1H, J=8.2, 1.1 Hz, H_{5′}), 8.06 (d, J=8.5 Hz, 1H, H_{4′}), 8.13 (d, 1H, J=8.5 Hz, H₈).

¹³C-RMN (CDCl₃, δ): 22.7, 24.9, 25.9, 26.3, 27.8 (—(CH₂)₂—, C₆, C₇, C₈), 38.2 (NCH₂), 39.3 (C₅), 48.7 (CH₂NH), 54.6 (CH₂Ar), 57.3 (C₈ₐ), 120.4 (C_{3′}), 126.3 (C_{6′}), 127.3, 127.5 (C_{5′}, C_{8′}), 128.9, 129.6 (C_{4′}, C_{7′}), 136.7 (C_{4′a}), 147.5 (C_{8′a}), 154.5 (C_{2′}), 158.0 (C₃), 173.2 (C₁).

Analysis calculated for C₂₁H₂₆N₄O₂.2HCl.½H₂O:

C, 56.25; H, 6.52; N, 12.50

Found: C, 56.66; H, 6.53; N, 11.94

### Example 38

### 2-[6-[(2-Quinolyl)methylamino]hexyl]-1,3-dioxoperhydroimidazo[1,5-a]pyridine (ll)

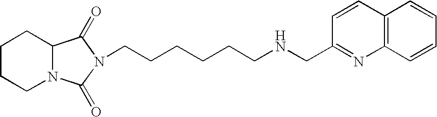

Chromatography: ethyl acetate/methanol, 7:3.

Yield: 30%; m.p. 176-188° C.

¹H-RMN (CDCl₃, δ): 1.19-1.74 (m, 12H, —(CH₂)₄—, H₆ₐₓ, H₇ₐₓ, H₈ₐₓ, H_{6ec}), 1.95-2.00 (m, 1H, H_{7ec}), 2.17-2.22 (m, 1H, H_{8ec}), 2.76 (t, 2H, J=7.1 Hz, CH₂NH), 2.81-2.86 (m, 1H, H₅ₐₓ), 3.06 (br s, 1H, NH), 3.48 (t, 2H, J=7.3 Hz, NCH₂), 3.72 (dd, 1H, J=11.9, 4.3 Hz, H₈ₐ), 4.14-4.19 (m, 3H, CH₂Ar, H_{5ec}), 7.46 (d, 1H, J=8.5 Hz, H_{3′}), 7.49-7.55 (m, 1H, H_{6′}), 7.68-7.74 (m, 1H, H_{7′}), 7.81 (dd, 1H, J=8.2, 1.1 Hz, H_{5′}), 8.06 (d, J=8.5 Hz, 1H, H_{4′}), 8.13 (d, 1H, J=8.4 Hz, H_{8′}).

¹³C-RMN (CDCl₃, δ): 22.7, 24.9, 26.3, 26.6, 27.8, 28.0, 29.0 (—(CH₂)₄—, C₆, C₇, C₈), 38.4 (NCH₂), 39.2 (C₅), 49.2 (CH₂NH), 54.6 (CH₂Ar), 57.2 (C₈ₐ), 120.3 (C_{3′}), 126.2 (C_{6′}), 127.3, 127.5 (C_{5′}, C_{8′}), 128.9, 129.6 (C_{4′}, C_{7′}), 136.7 (C_{4′a}), 147.5 (C_{8′a}), 154.5 (C_{2′}), 158.2 (C₃), 173.2 (C₁).

Analysis calculated for C₂₃H₃₀N₄O₂.2HCl.3/2H₂O:

C, 55.87; H, 7.13; N, 11.33

Found: C, 55.77; H, 7.09; N, 10.77

### Example 39

### Radioligand Binding Assays

The in vitro affinity of the compounds of the present invention for the 5-HT_{1A}, 5-HT_{2A}, 5-HT₃, 5-HT₄, 5-HT₇, α₁ and D₂ cerebral receptors was evaluated using radioligand binding assays. The following specific ligands and tissues were used:
5-HT_{1A} receptors, [³H]-8-OH-DPAT, rat cerebral cortex;
5-HT_{2A} receptors, [³H]ketanserin, rat cerebral cortex;
5-HT₃ receptors, [³H]LY 278584, rat cerebral cortex;
5-HT₄ receptors, [³H]GR 113808, rat striatum;
5-HT₇ receptors, [³H]-5-CT, rat hypothalamus;
α₁ receptors, [³H]prazosin, rat cerebral cortex;
D₂ receptors, [³H]spiperone, rat striatum.

Compound BAYx3702 was selected as a 5-HT_{1A} reference ligand, as well as the left-hand isomer of the same, (−)-BAYx3702.

For all receptor binding assays, male Sprague-Dawley rats (Rattus norvegicus albinus), weighing 180-200 g, were killed by decapitation and the brains rapidly removed and dissected. Tissues were stored at −80° C. for subsequent use and homogenized on a Polytron PT-10 homogenizer. Membrane suspensions were centrifuged on a Beckman J2-HS instrument.

The bonded radioactive ligands were separated from the free ones by vacuum filtration on Whatman GF/C filters washed twice with 4 mL of the corresponding buffer. 4 mL of liquid scintillation (EcoLite) were added and the radioactivity bonded to the membranes was measured by liquid scintillation spectrometry.

### 5-HT_{1A} Receptor

Binding assays were performed by a modification of the procedure previously described by Clark et al. (J. Med. Chem., 1990, 33, 633), as described below.

The cerebral cortex was homogenized in 10 volumes of ice-cold Tris buffer (50 mM Tris-HCl, pH 7.7 at 25° C.) and centrifuged at 28000 g for 15 min. The membrane pellet was washed twice by resuspension and centrifugation. After the second wash the resuspended pellet was incubated at 37° C. for 10 min. Membranes were then collected by centrifugation and the final pellet was resuspended in 50 mM Tris-HCl, 5 mM MgSO₄, and 0.5 mM EDTA buffer (pH 7.4 at 37° C.). Fractions of 100 μL of the final membrane suspension (about 1 mg of protein) were incubated at 37° C. for 15 min with 0.6 nM [³H]-8-OH-DPAT (133 Ci/mmol), in the presence or absence of the competing drug, in a final volume of 1.1 mL of assay buffer (50 mM Tris-HCl, 10 nM clonidine, 30 nM prazosin, pH 7.4 at 37° C.). Nonspecific binding was determined with 10 μM 5-HT.

### 5-HT_{2A} Receptor

Binding assays were performed by a modification of the procedure previously described by Titeler et al. (Biochem. Pharmacol., 1987, 36, 3265), as described below.

The frontal cortex was homogenized in 60 volumes of ice-cold buffer (50 mM Tris-HCl, 0.5 mM Na₂EDTA, 10 mM MgSO₄, pH 7.4 at 25° C.), and centrifuged at 30000 g for 15 min at 4° C. The membrane pellet was washed by resuspension and centrifugation. After the second wash the resuspended pellet was incubated at 37° C. for 10 min. Membranes were then collected by centrifugation and the final pellet was resuspended in 10 volumes of assay buffer (50 mM Tris-HCl, 0.5 mM Na₂EDTA, 10 mM MgSO₄, 0.1% ascorbic acid, 10 μM pargyline, pH 7.4 at 25° C.). Fractions of 100 μL of the final membrane suspension (about 5 mg/mL of protein) were incubated at 37° C. for 15 min with 0.4 nM [³H]ketanserin, in the presence or absence of the competing drug, in a final volume of 2 mL of assay buffer. Nonspecific binding was determined with 1 μM cinanserin.

### 5-HT₃ Receptor

Binding assays were performed by a modification of the procedure previously described by Wong et al. (Eur. J. Pharmacol., 1989, 166, 107), as described below.

The cerebral cortex was homogenized in 9 volumes of ice-cold 0.32 M sucrose and centrifuged at 1000 g for 10 min at 4° C. The supernatant was centrifuged at 17000 g for 20 min at 4° C. The membrane pellet was washed twice by resuspension in 60 volumes of ice-cold 50 mM Tris-HCl buffer (pH 7.4 at 25° C.) and centrifugation at 48000 g for 10 min at 4° C. After the second wash the resuspended pellet was incubated at 37° C. for 10 min, and centrifuged at 48000 g for 10 min at 4° C. Membranes were resuspended in 2.75 volumes of assay buffer (50 mM Tris-HCl, 10 μM pargyline, 0.6 mM ascorbic acid, and 5 mM CaCl₂, pH 7.4 at 25° C.). Fractions of 100 μL of the final membrane suspension (about 2 mg/mL of protein) were incubated at 25° C. for 30 min with 0.7 nM [³H]LY 278584, in the presence or absence of the competing drug, in a final volume of 2 mL of assay buffer. Nonspecific binding was determined with 10 μM 5-HT.

### 5-HT₄ Receptor

Binding assays were performed by a modification of the procedure previously described by Grossman et al. (Br. J. Pharmacol., 1993, 109, 618), as described below.

The striatum was homogenized in 15 volumes of ice-cold 50 mM HEPES buffer (pH 7.4 at 4° C.) and centrifuged at 48000 g for 10 min. The pellet was resuspended in 20 volumes of assay buffer (50 mM HEPES, pH 7.4 at 25° C.). Fractions of 100 μL (about 5 mg/mL of protein) of the final membrane suspension were incubated at 37° C. for 30 min with 0.1 nM [³H]GR 113808, in the presence or absence of the competing drug, in a final volume of 1 mL of assay buffer. Nonspecific binding was determined with 30 μM 5-HT.

### 5-HT₇ Receptor

Binding assays were performed by a modification of the procedure previously described by Aguirre et al. (Eur. J. Pharmacol., 1998, 346, 181), as described below.

The hypothalamus was homogenized in 5 mL of ice-cold Tris buffer (50 mM Tris-HCl, pH 7.4 at 25° C.) and centrifuged at 48000 g for 10 min. The membrane pellet was washed by resuspension and centrifugation, and then the resuspended pellet was incubated at 37° C. for 10 min. Membranes were then collected by centrifugation and the final pellet was resuspended in 100 volumes of ice-cold 50 mM Tris-HCl, 4 mM CaCl₂, 1 mg/mL ascorbic acid, 0.01 mM pargyline and 3 μM pindolol buffer (pH 7.4 at 25° C.). Fractions of 400 μL of the final membrane suspension were incubated at 23° C. for 120 min. with 0.5 nM [³H]-5-CT (88 Ci/mmol), in the presence or absence of several concentrations of the competing drug, in a final volume of 0.5 mL of assay buffer (50 mM Tris-HCl, 4 mM CaCl₂, 1 mg/mL ascorbic acid, 0.01 mM pargyline and 3 μM pindolol buffer (pH 7.4 at 25° C.)). Non-specific binding was determined with 10 μM 5-HT.

### α₁ Receptor

Binding assays were performed by a modification of the procedure previously described by Ambrosio et al. (Neurosci. Lett., 1984, 49, 193), as described below.

The cerebral cortex was homogenized in 20 volumes of ice-cold buffer (50 mM Tris-HCl, 10 mM MgCl₂, pH 7.4 at 25° C.) and centrifuged at 30000 g for 15 min. Pellets were washed twice by resuspension and centrifugation. Final pellets were resuspended in the same buffer. Fractions of the final membrane suspension (about 250 μg of protein) were incubated at 25° C. for 30 min with 0.2 nM [³H]prazosin (23 Ci/mmol), in the presence or absence of six concentrations of the competing drug, in a final volume of 2 mL of buffer. Nonspecific binding was determined with 10 μM phentolamine.

### D₂ Receptor

Binding assays were performed by a modification of the procedure previously described by Leysen et al. (Biochem. Pharmacol., 1978, 27, 307), as described below.

The striatum was homogenized in 50 mM Tris-HCl (pH 7.7 at 25° C.) and centrifuged at 48000 g for 10 min. The pellet was resuspended and centrifuged as before. The final pellet was resuspended in 50 mM Tris-HCl (pH 7.7 at 25° C.) containing 120 mM NaCl, 5 mM KCl, 2 mM CaCl₂, 1 mM MgCl₂, and 0.1% ascorbic acid. Fractions of the final membrane suspension (125-150 μg of protein) were incubated at 25° C. for 60 min with 0.8 nM [³H]raclopride (77 Ci/mmol), in the presence or absence of six concentrations of the competing drug, in a final volume of 1.1 mL of the assay buffer (pH 7.4 at 25° C.). Nonspecific binding was determined with 1 μM (+)-butaclamol.

For all binding assays, competing drug, nonspecific, total and radioligand bindings were defined in triplicate. Incubation was terminated by rapid vacuum filtration through Whatman GF/B filters, presoaked in 0.05% poly(ethylenimine), using a Brandel cell harvester. The filters were then washed with the assay buffer, dried and placed in poly(ethylene) vials to which were added 4 mL of a scintillation cocktail (Aquasol). The radioactivity bound to the filters was measured by liquid scintillation spectrometry. The data were analyzed by an iterative curve-fitting procedure (program Prism, Graph Pad), which provided IC₅₀, Kᵢ, and r² values for test compounds, Kᵢ values being calculated from the Cheng and Prusoff equation. The protein concentrations of the rat cerebral cortex and the rat striatum were determined by the method of Lowry, using bovine serum albumin as the standard.

### Results:

All the tested compounds showed a high affinity for the 5-HT_{1A} receptor with a Kᵢ value of between 0.5 and about 100 nM. Most of the compounds bind the 5-HT_{1A} receptor with an affinity of below 30 nM. Also, most of the compounds are highly selective for the 5-HT_{1A} receptor over 5-HT_{2A} 5-HT₃, 5-HT₄ and dopamine receptors.

### Example 40

### Functional Characterization

Cell Culture and Determination of cAMP Levels After Stimulation of the Adenylate Cyclase Enzyme with Forskolin.

HeLa cells transfected with the human 5-HT_{1A} receptor (HA 6 cells) were grown in 75 mL flasks containing 20 mL of Dulbecco's modified Eagle's medium (DMEM) supplemented with 10% fetal calf serum, 500 units penicillin and 500 μg streptomycin/mL (P/S) and 0.3 mg/mL geneticin. Forty-eight hours before the experiment cells were plated at a density of 75×10³ cells in 1.5 mL of DMEM-P/S-fetal calf serum-geneticin medium in 12 multiwell plates. The day of the experiment, cells were treated with 0.5 mM 1-methyl-3-isobutylxanthine (IBMX), 10 μM forskolin, and vehicle or and different concentrations of the compounds under study in a 37° C. and 5% CO₂ incubator. Ten minutes later, treatment was stopped and cells were lysed with a 65% ethanol solution for 2 h, then the ethanol was collected and evaporated at 55° C. leaving a pellet with cAMP. Samples were analyzed using a commercial radioimmunoassay (RIA) kit ([³H]CAMP assay system, cod. TRK 432; Amersham). Protein was measured by Bradford's method. Competition binding isotherms were analysed by using an iterative curve-fitting procedure (program Origin 7.0), which provided EC₅₀ values for test compounds.

The experiment shows that the tested compounds significantly inhibit the AMPc formation in HeLa cells, most compounds acting as pure and some as partial agonists.

### Determination of the Rectal Temperature in Mice.

5-HT_{1A} receptor agonists such as, for example, 8-OH-DPAT, reduce the body temperature of the rodents. This effect in the mouse seems to be due to the activation of the somatodendritic receptors (De Vry, Psychopharmacology 1995, 121, 1) since the administration for two weeks of a tryptophan hydroxylase inhibitor such as parachlorophenylalanine or the damage with a selective neurotoxin of serotoninergic neurons such as 5,7-dihydroxytryptamine (5,7-DHT) completely blocks the hypothermic effect on mice.

In the test carried out with the compounds of the present invention, batches of 8-10 mice were processed, testing at least 4 doses of the compounds object under study. Animals were maintained in a temperature and light (25±1° C., light on between 8.00 a.m. and 8.00 p.m.) controlled environment. Food and tap water were provided ad libitum. All experiments were performed between 9.00 a.m. and 2.00 p.m. The test consisted of inserting a probe into the animals rectum 1.5 cm for 40 s measuring the basal temperature, this being the 0 time of the experiment. Immediately afterwards, the compounds to be tested were administered subcutaneously (s.c) and the rectal temperature was measured after different times: 15, 30, 60, 120 and 240 minutes.

The experiment shows that the tested compounds are 5-HT_{1A} agonists, according to the minimum effective dose administered and the hypothermic effect obtained.

### Example 41

### In Vitro Neuroprotection Studies

### Neurotoxicity Induced by Hypoxia/Hypoglycaemia

The capacity to prevent neurotoxicity induced by hypoxia/hypoglycaemia in primary cultures of rat hippocampus (E18) was determined. To prepare the cultures, the foetuses' brain was dissected, separating the meninges, and the hippocampus was dispersed on a neurobasal medium supplemented with B-27. After centrifuging at 700 g, the pellet was mechanically redispersed. The density of the cellular suspension was measured and aliquots were taken to culture on Petri dishes, previously coated with poly-lysine, using the same medium. The culture was kept in an incubator at 37° C. in a 95% air/5% CO₂ atmosphere. After 10 days' culture, the dishes were transferred, in a glucose-free medium, to a chamber wherein they were kept for 2 hours in a 95% N₂/5% CO₂ atmosphere. Before the hypoxia, the compounds to be studied were added at variable times and concentrations.

### Neurotoxicity Due to Deprivation of Trophic Factors.

The prevention of cellular death with apoptotic characteristics which results after maintaining the hippocampus cultures in a serum-free medium for 48 hours (Koh et al., Science, 1995, 268, 573) was also studied. In this case, an Eagle medium (DMEM) modified with 10% of calf serum was initially used and, after 10 days, the cultures were transferred to the DMEM culture deprived of calf serum.

In both cases, the measurement of the mitochondrial dehydrogenase on 3-(4,5-dimethylthiazol-2-yl)-2,5-diphenyltetrazolium bromide (MTT) with subsequent calorimetric measurement of the formazan formed, which provides a cell survival index (Nonaka et al., Proc. Natl. Acad. Sci. USA, 1998, 95, 2642), was considered as toxicity indexes.

The results obtained are set down in the following table 1.

TABLE 1

Protection (%)

| | | Oxygen-glucose |
|---|---|---|
| Compound | Serum Deprivation | deprivation |
| 8-OH-DPAT | 44.0 ± 1.4 | 54.0 ± 3.5 |
| (−)-BAYx3702 | 31.0 ± 2.9 | 77.5 ± 8.6 |
| (±)-BAYx3702 | 25.9 ± 2.5 | ND |
| a | 10.7 ± 2.4 | 55.8 ± 12.5 |
| b | 19.0 ± 3.3 | 42.0 ± 7.8 |
| c | 13.0 ± 4.3 | 65.6 ± 4.8 |
| d | 15.5 ± 2.2 | 78.0 ± 6.5 |
| e | 29.6 ± 2.8 | 78.0 ± 6.8 |
| f | 36.0 ± 3.2 | 35.0 ± 7.0 |
| g | 32.0 ± 4.0 | 32.0 ± 8.0 |

### Example 42

### In Vivo Neuroprotection Study

### Focal Ischemic Model in Rats.

The intraluminal occlusion of the middle cerebral artery (MCA) in rats was performed following previously described methods (Justicia et al., J Cereb. Blood Flow Metab., 1999, 19, 128), as described below.

The rats were anaesthetised with halotan, maintaining their temperature at 37.5° C. using an electric blanket connected to a rectal probe and cannulating the left femoral artery to monitor blood pressure. The right carotid artery was exposed, occluding the extra-cranial branches, and a blunt nylon filament was introduced through the external carotid until reaching the level when the middle cerebral artery (MCA) branches.

The selected compounds were administered intravenously. 24 Hours after the ischemic damage, the rats were anaesthetised with ether, they were perfused with saline solution and were then decapitated, extracting the brains that were cut into 1.5 mm coronal slices. The slices were then incubated in 4% triphenyltetrazolium chloride (TTC), which reacts with the intact mitochondrial enzymes producing a red colour which contrasts with the paleness of the infracted area, permitting it to be viewed, subsequently maintaining it in 10% formalin. The volume of cerebral infarction (mm³) was calculated by measuring, in an image analyser, the affected area in the areas of cerebral cortex irrigated by the MCA and in the striatum and multiplying the average value obtained in each slice by the thickness thereof.

The intravenous infusion of 40 μg/Kg of compound (e) for 4 hours significantly reduced the infracted cerebral volume in the rat after intraluminal occlusion of the MCA. The effect was observed even when the rats received the compound for a period of 2 hours after the start of the MCA occlusion. The protective effect was only apparent in the cortical area but not in the subcortical area and did not cause alterations in the arterial blood pressure.

In Table 2, the values of infracted volume both for a saline solution, and for the standard (−)-BAYx3702 and for compound (e), are set down.

TABLE 2

| | | Infarcted Volumen (mm³) | | |
|---|---|---|---|---|
| Compound | Dose | Total | Cortical | Subcortical |
| Saline | — | 546.4 ± 51.1 | 399.2 ± 37.5 | 147.2 ± 23.4 |
| (−)-BAYx3702 | 40 | 376.7 ± 57.8* | 271.2 ± 54.7* | 105.5 ± 9.3 |
| | μg/kg | | | |
| e | 40 | 386.7 ± 47.1* | 262.7 ± 40.9* | 124.0 ± 14.3 |
| | μg/kg | | | |

*p < 0.05 vs. Control (saline)

1. A compound, a stereochemical isomer of the compound, or a hydrate, solvate, or pharmaceutically acceptable salt of the compound or isomer, wherein:
the compound corresponds in structure to formula I: 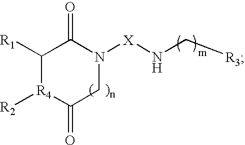
R₄ is selected from the group consisting of N and S;
if R₄ is S, then R₁ is H, and R₂ is absent;
if R₄ is N, then R₁ and R₂ are H or are methylene groups bound together forming with the heterocyclic ring a 5- or 6-membered ring;
n is zero or 1;
X is selected from the group consisting of C₂-C₁₀-alkylene, C₂-C₁₀-alkenylyne, and —CH₂—Y—CH₂—;
Y is phenyl;
m is 1 or 2;
R₃ is selected from the group consisting of chroman-2-yl, 2-quinolyl, and phenoxy, wherein:
the quinolyl, the aromatic ring of the chromanyl, and the phenyl ring of the phenoxy are optionally substituted with one or more substituents independently selected from the group consisting of C₁-C₆-alkoxy, C₁-C₆-alkyl, halogen, C₂-C₆-alkenyl, halo-(C₁-C₆)-alkyl, halo-(C₁-C₆)-alkoxy, phenyl, phenyl(C₁-C₆)-alkyl, phenoxy, C₁-C₆-alkylcarbonyl, phenylcarbonyl, phenyl(C₁-C₆)alkylcarbonyl, C₁-C₆-alkoxycarbonyl, phenyl(C₁-C₆)alkoxycarbonyl, C₁-C₆-alkyl-carbonylamino, hydroxy, cyano, nitro, amino, N—(C₁-C₆)-alkylamino, N,N—(C₁-C₆)-dialkylamino, carboxy, sulfo, sulfamoyl, sulfonylamino, (C₁-C₆)alkylaminosulfonyl, and (C₁-C₆)alkylsulfonylamino, wherein:
the C₁-C₆-alkyl portion of any of the alkyl-comprising substituents is optionally substituted with a substituent independently selected from the group consisting of hydroxy and amino; or
the phenyl ring of the phenoxy is substituted by two neighbouring residues, which together with the phenyl ring to which they are attached form tetrahydronaphthyl;
provided that the compound is not
2-[4-[(chroman-2-yl)methylamino]butyl]-1,3-dioxoperhydropyrrolo[1,2-c]imidazole,
3-[4-[(chroman-2-yl)methylamino]butyl]-2,4-dioxothiazolidine,
3-[5-[(chroman-2-yl)methylamino]pentyl]-2,4-dioxothiazolidine,
3-[6-[(chroman-2-yl)methylamino]hexyl]-2,4-dioxothiazolidine,
2-[4-[2-(phenoxy)ethylamino]butyl]-1,3-dioxoperhydropyrrolo[1,2-c]imidazole,
3-[4-[2-(phenoxy)ethylamino]butyl]-2,4-dioxothiazolidine, or
3-[3-[(chroman-2-yl)methylamino]propyl]-2,4-dioxoimidazolidine.

2. The compound, isomer, hydrate, solvate, or salt according to claim 1, wherein R₃ is selected from the group consisting of chroman-2-yl, 2-quinolyl, and phenoxy, wherein
the phenyl ring of the phenoxy is optionally substituted with a substituent selected from the group consisting of C₁-C₆-alkoxy, C₁-C₆-alkyl, and halogen.

3. The compound, isomer, hydrate, solvate, or salt according to claim 1, wherein:
m is 1; and
R₃ is chroman-2-yl.

4. The compound, isomer, hydrate, solvate, or salt according to claim 3, wherein:
R₁ and R₂ are methylene groups bound together forming with the heterocyclic ring a 5- or 6-membered ring; and
R₄ is N.

5. The compound, isomer, hydrate, solvate, or salt according to claim 3, wherein X is selected from the group consisting of C₂-C₁₀-alkylene, (E)-2-butenylene,

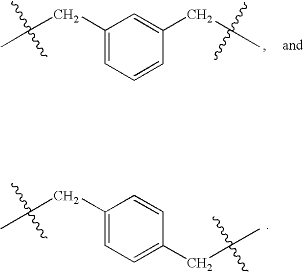

6. The compound, isomer, hydrate, solvate, or salt according to claim 3, wherein:
R₁ is H;
R₂ is absent; and
R₄ is S.

7. The compound, isomer, hydrate, solvate, or salt according to claim 6, wherein:
n is zero; and
X is C₂-C₁₀-alkylene.

8. The compound, isomer, hydrate, solvate, or salt according to claim 1, wherein:
m is 2; and
R₃ is phenoxy, wherein the phenyl ring of the phenoxy:
is optionally substituted with one or more substituents independently selected from the group consisting of C₁-C₆-alkoxy, C₁-C₆-alkyl, halogen, C₂-C₆-alkenyl, halo-(C₁-C₆)-alkyl, halo-(C₁-C₆)-alkoxy, phenyl, phenyl(C₁-C₆)-alkyl, phenoxy, C₁-C₆-alkylcarbonyl, phenylcarbonyl, phenyl(C₁-C₆)alkylcarbonyl, C₁-C₆-alkoxycarbonyl, phenyl(C₁-C₆)alkoxycarbonyl, C₁-C₆-alkylcarbonylamino, hydroxy, cyano, nitro, amino, N—(C₁-C₆)-alkylamino, N,N—(C₁-C₆)-dialkylamino, carboxy, sulfo, sulfamoyl, sulfonylamino, (C₁-C₆)alkylaminosulfonyl, and (C₁-C₆)alkylsulfonylamino; or
is substituted by two neighbouring residues, which together with the phenyl ring to which they are attached form tetrahydronaphthyl.

9. The compounds isomer, hydrate, solvate, or salt according to claim 8, wherein:
R₃ is phenoxy, wherein the phenyl ring of the phenoxy:
is optionally substituted with one or more substituents independently selected from the group consisting of phenyl, C₁-C₆-alkoxycarbonyl, C₁-C₆-alkylcarbonylamino, C₁-C₆-alkoxy, C₁-C₆-alkyl, halo-(C₁-C₆)-alkyl, and halogen, or
is substituted by two neighbouring residues, which together with the phenyl group to which they are attached form tetrahydronaphthyl.

10. The compound, isomer, hydrate, solvate, or salt according to claim 9, wherein the phenyl ring of the phenoxy is optionally substituted with one or more substituents independently selected from the group consisting of methoxy, ethoxy, propoxy, isopropoxy, ethyl, propyl, isopropyl, bromide, trifluoromethyl, methylamido, and ethoxycarbonyl.

11. The compound, isomer, hydrate, solvate, or salt according to claim 8, wherein R₃ is phenoxy, wherein:
the phenyl ring of the phenoxy is substituted in ortho- and/or meta-position.

12. The compound, isomer, hydrate, solvate, or salt according to claim 8, wherein:
R₁ and R₂ are methylene groups bound together forming with the heterocyclic ring a 5- or 6-membered ring; and
R₄ is N.

13. The compound, isomer, hydrate, solvate, or salt according to claim 8, wherein:
n is zero; and
X is C₂-C₁₀-alkylene.

14. The compound, isomer, hydrate, solvate, or salt according to claim 8, wherein:
R₁ is H;
R₂ is absents and
R₄ is S.

15. The compound, isomer, hydrate, solvate, or salt according to claim 14, wherein:
n is zero; and
X is C₂-C₁₀-alkylene.

16. The compound, isomer, hydrate, solvate, or salt according to claim 1, wherein:
m is 1; and
R₃ is 2-quinolyl.

17. The compound, isomer, hydrate, solvate, or salt according to claim 16, wherein:
R₁ and R₂ are methylene groups bound together forming with the heterocyclic ring a 5- or 6-membered ring; and
R₄ is N.

18. The compound, isomer, hydrate, solvate, or salt according to claim 16, wherein:
n is zero; and
X is C₂-C₁₀-alkylene.

19. The compound, isomer, hydrate, solvate, or salt according to claim 1, wherein the compound is selected from the group consisting of:
2-[4-[(Chroman-2-yl)methylamino]butyl]-1,3-dioxoperhydroimidazo[1,5-a]pyridine;
2-[4-[(Chroman-2-yl)methylamino]butyl]-1,3-dioxoperhydropyrrolo[1,2-a]pyrazine;
2-[5-[(Chroman-2-yl)methylamino]pentyl]-1,3-dioxoperhydropyrrolo[1,2-c]imidazole;
2-[6-[(Chroman-2-yl)methylamino]hexyl]-1,3-dioxoperhydropyrrolo[1,2-c]imidazole;
2-[3-[(Chroman-2-yl)methylamino]propyl]-1,3-dioxoperhydropyrrolo[1,2-c]imidazole;
3-[8-[(Chroman-2-yl)methylamino]octyl]-2,4-dioxothiazolidine;
2-[4-[(Chroman-2-yl)methylamino]butyl]-1,3-dioxoperhydropyrrolo[1,2-c]imidazole;
2-[8-[(Chroman-2-yl)methylamino]octyl]-1,3-dioxoperhydropyrrolo[1,2-c]imidazole;
2-[3-[[(Chroman-2-yl)methylamino]methyl]benzyl]-1,3-dioxoperhydropyrrolo[1,2-c]imidazole;
2-[4-[[(Chroman-2-yl)methylamino]methyl]benzyl]-1,3-dioxoperhydropyrrolo[1,2-c]imidazole;
2-[4-[(Chroman-2-yl)methylamino]but-2-enyl]-1,3-dioxoperhydropyrrolo[1,2-c]imidazole;
2-[4-[2-(o-Methoxyphenoxy)ethylamino]butyl]-1,3-dioxoperhydropyrrolo[1,2-c]imidazole;
2-[4-[2-(m-Methoxyphenoxy)ethylamino]butyl]-1,3-dioxoperhydropyrrolo[1,2-c]imidazole;
2-[4-[2-(o-Bromophenoxy)ethylamino]butyl]-1,3-dioxoperhydropyrrolo[1,2-c]imidazole;
2-[4-[2-(m-Bromophenoxy)ethylamino]butyl]-1,3-dioxoperhydropyrrolo[1,2-c]imidazole;
2-[4-[2-(o-Ethylphenoxy)ethylamino]butyl]-1,3-dioxoperhydropyrrolo[1,2-c]imidazole;
2-[4-[2-(m-Ethylphenoxy)ethylamino]butyl]-1,3-dioxoperhydropyrrolo[1,2-c]imidazole;
2-[4-[2-(o-Isopropylphenoxy)ethylamino]butyl]-1,3-dioxoperhydropyrrolo[1,2-c]imidazole;
2-[4-[(2-quinolyl)methylamino]butyl]-1,3-dioxoperhydropyrrolo[1,2-c]imidazole;
2-[4-[2-(o-Ethoxyphenoxy)ethylamino]butyl]-1,3-dioxoperhydropyrrolo[1,2-c]imidazole;
2-[4-[2-(o-Isopropoxyphenoxy)ethylamino]butyl]-1,3-dioxoperhydropyrrolo[1,2-c]imidazole;
2-[4-[2-[m-(Trifluoromethyl)phenoxy]ethylamino]butyl]-1,3-dioxoperhydropyrrolo[1,2-c]imidazole;
2-[4-[2-(1,1′-Biphenyl-2-yloxy)ethylamino]butyl]-1,3-dioxoperhydropyrrolo[1,2-c]imidazole;
2-[4-[2-[o-(Acetylamino)phenoxy]ethylamino]butyl]-1,3-dioxoperhydropyrrolo[1,2-c]imidazole;
2-[4-[2-[m-(Acetylamino)phenoxy]ethylamino]butyl]-1,3-dioxoperhydropyrrolo[1,2-c]imidazole;
2-[4-[2-[o-(Ethoxycarbonyl)phenoxy]ethylamino]butyl]-1,3-dioxoperhydropyrrolo[1,2-c]imidazole;
2-[4-[2-(5,6,7,8-Tetrahydronaphth-1-yloxy)ethylamino]butyl]-1,3-dioxoperhydropyrrolo[1,2-c]imidazole;
2-[4-[2-(2,3-Dimethylphenoxy)ethylamino]butyl]-1,3-dioxoperhydropyrrolo[1,2-c]imidazole;
2-[4-[(Chroman-2-yl)methylamino]butyl]-1,4-dioxoperhydropyrido[1,2-a]pyrazine;
2-[4-[(Chroman-2-yl)methylamino]but-2-enyl]-1,4-dioxoperhydropyrrolo[1,2-c]imidazole;
3-[4-[2-(o-Ethoxyphenoxy)ethylamino]butyl]-2,4-dioxothiazolidine;
3-[6-[2-(o-Ethoxyphenoxy)ethylamino]hexyl]-2,4-dioxothiazolidine;
3-[8-[2-(o-Ethoxyphenoxy)ethylamino]octyl]-2,4-dioxothiazolidine;
2-[4-[2-(o-Ethoxyphenoxy)ethylamino]butyl]-1,3-dioxoperhydroimidazo[1,5-a]pyridine;
2-[6-[2-(o-Ethoxyphenoxy)ethylamino]hexyl]-1,3-dioxoperhydroimidazo[1,5-a]pyridine;
2-[4-[(2-Quinolyl)methylamino]butyl]-1,3-dioxoperhydroimidazo[1,5-a]pyridine; and
2-[6-[(2-Quinolyl)methylamino]hexyl]-1,3-dioxoperhydroimidazo[1,5-a]pyridine.

20. A pharmaceutical composition which comprises:
a therapeutically effective amount of a compound, a stereochemical isomer of the compound, or a hydrate, solvate, or pharmaceutically acceptable salt of the compound or isomer, wherein the compound is selected from the group of compounds recited in claim 1; and
one or more pharmaceutically acceptable carriers.

21. A use of a compound, a stereochemical isomer of the compound, or a hydrate, solvate, or pharmaceutically acceptable salt of the compound or isomer for the preparation of a medicament for the treatment and/or prophylaxis of a condition selected from the group consisting of Parkinson Disease, cerebral damage by thromboembolitic ictus, craneoencephalic traumatisms, depression, migraine, pain, psychosis, anxiety disorders, aggressive disorders, and urinary tract disorders, wherein:
the compound corresponds in structure to formula I: 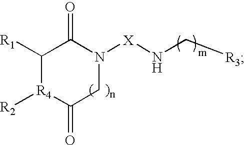
R₄ is selected from the group consisting of N and S;
if R₄ is S, then R₁ is H, and R₂ is absent;
if R₄ is N, then R₁ and R₂ are H or are methylene groups bound together forming with the heterocyclic ring a 5- or 6-membered ring;
n is zero or 1;
X is selected from the group consisting of C₂-C₁₀-alkylene, C₂-C₁₀-alkenylyne, and —CH₂—Y—CH₂—;
Y is phenyl;
m is 1 or 2;
R₃ is selected from the group consisting of chroman-2-yl, 2-quinolyl, and phenoxy, wherein:
the quinolyl, the aromatic ring of the chromanyl, and the phenyl ring of the phenoxy are optionally substituted with one or more substituents independently selected from the group consisting of C₁-C₆-alkoxy, C₁-C₆-alkyl, halogen, C₂-C₆-alkenyl, halo-(C₁-C₆)-alkyl, halo-(C₁-C₆)-alkoxy, phenyl, phenyl(C₁-C₆)-alkyl, phenoxy, C₁-C₆-alkylcarbonyl, phenylcarbonyl, phenyl(C₁-C₆)alkylcarbonyl, C₁-C₆-alkoxycarbonyl, phenyl(C₁-C₆)alkoxycarbonyl, C₁-C₆-alkyl-carbonylamino, hydroxy, cyano, nitro, amino, N—(C₁-C₆)-alkylamino, N,N—(C₁-C₆)-dialkylamino, carboxy, sulfo, sulfamoyl, sulfonylamino, (C₁-C₆)alkylaminosulfonyl, and (C₁-C₆)alkylsulfonylamino, wherein:
the C₁-C₆-alkyl portion of any of the alkyl-comprising substituents is optionally substituted with a substituent independently selected from the group consisting of hydroxy and amino; or
the phenyl ring of the Phenoxy is substituted by two neighbouring residues, which together with the phenyl ring to which they are attached form tetrahydronaphthyl:
provided that the compound is not
2-[4-[(chroman-2-yl)methylamino]butyl]-1,3-dioxoperhydropyrrolo[1,2-c]imidazole,
3-[4-[(chroman-2-yl)methylamino]butyl]-2,4-dioxothiazolidine,
3-[5-[(chroman-2-yl)methylamino]pentyl]-2,4-dioxothiazolidine,
3-[6-[(chroman-2-yl)methylamino]hexyl]-2,4-dioxothiazolidine,
2-[4-[2-(phenoxy)ethylamino]butyl]-1,3-dioxoperhydropyrrolo[1,2-c]imidazole, or
3-[4-[2-(Phenoxy)ethylamino]butyl]-2,4-dioxothiazolidine.

22. The use according to claim 21, wherein the compound is selected from the group consisting of:
2-[4-[(Chroman-2-yl)methylamino]butyl]-1,3-dioxoperhydroimidazo[1,5-a]pyridine;
2-[4-[(Chroman-2-yl)methylamino]butyl]-1,3-dioxoperhydropyrrolo[1,2-a]pyrazine;
2-[5-[(Chroman-2-yl)methylamino]pentyl]-1,3-dioxoperhydropyrrolo[1,2-c]imidazole;
2-[6-[(Chroman-2-yl)methylamino]hexyl]-1,3-dioxoperhydropyrrolo[1,2-c]imidazole;
2-[3-[(Chroman-2-yl)methylamino]propyl]-1,3-dioxoperhydropyrrolo[1,2-c]imidazole;
3-[8-[(Chroman-2-yl)methylamino]octyl]-2,4-dioxothiazolidine;
2-[4-[(Chroman-2-yl)methylamino]butyl]-1,3-dioxoperhydropyrrolo[1,2-c]imidazole;
2-[8-[(Chroman-2-yl)methylamino]octyl]-1,3-dioxoperhydropyrrolo[1,2-c]imidazole;
2-[3-[[(Chroman-2-yl)methylamino]methyl]benzyl]-1,3-dioxoperhydropyrrolo[1,2-c]imidazole;
2-[4-[[(Chroman-2-yl)methylamino]methyl]benzyl]-1,3-dioxoperhydropyrrolo[1,2-c]imidazole;
2-[4-[(Chroman-2-yl)methylamino]but-2-enyl]-1,3-dioxoperhydropyrrolo[1,2-c]imidazole;
2-[4-[2-(o-Methoxyphenoxy)ethylamino]butyl]-1,3-dioxoperhydropyrrolo[1,2-c]imidazole;
2-[4-[2-(m-Methoxyphenoxy)ethylamino]butyl]-1,3-dioxoperhydropyrrolo[1,2-c]imidazole;
2-[4-[2-(o-Bromophenoxy)ethylamino]butyl]-1,3-dioxoperhydropyrrolo[1,2-c]imidazole;
2-[4-[2-(m-Bromophenoxy)ethylamino]butyl]-1,3-dioxoperhydropyrrolo[1,2-c]imidazole;
2-[4-[2-(o-Ethylphenoxy)ethylamino]butyl]-1,3-dioxoperhydropyrrolo[1,2-c]imidazole;
2-[4-[2-(m-Ethylphenoxy)ethylamino]butyl]-1,3-dioxoperhydropyrrolo[1,2-c]imidazole;
2-[4-[2-(o-Isopropylphenoxy)ethylamino]butyl]-1,3-dioxoperhydropyrrolo[1,2-c]imidazole;
2-[4-[(2-quinolyl)methylamino]butyl]-1,3-dioxoperhydropyrrolo[1,2-c]imidazole;
2-[4-[2-(o-Ethoxyphenoxy)ethylamino]butyl]-1,3-dioxoperhydropyrrolo[1,2-c]imidazole;
2-[4-[2-(o-Isopropoxyphenoxy)ethylamino]butyl]-1,3-dioxoperhydropyrrolo[1,2-c]imidazole;
2-[4-[2-[m-(Trifluoromethyl)phenoxy]ethylamino]butyl]-1,3-dioxoperhydropyrrolo[1,2-c]imidazole;
2-[4-[2-(1,1′-Biphenyl-2-yloxy)ethylamino]butyl]-1,3-dioxoperhydropyrrolo[1,2-c]imidazole;
2-[4-[2-[o-(Acetylamino)phenoxy]ethylamino]butyl]-1,3-dioxoperhydropyrrolo[1,2-c]imidazole;
2-[4-[2-[m-(Acetylamino)phenoxy]ethylamino]butyl]-1,3-dioxoperhydropyrrolo[1,2-c]imidazole;
2-[4-[2-[o-(Ethoxycarbonyl)phenoxy]ethylamino]butyl]-1,3-dioxoperhydropyrrolo[1,2-c]imidazole;
2-[4-[2-(5,6,7,8-Tetrahydronaphth-1-yloxy)ethylamino]butyl]-1,3-dioxoperhydropyrrolo[1,2-c]imidazole;
2-[4-[2-(2,3-Dimethylphenoxy)ethylamino]butyl]-1,3-dioxoperhydropyrrolo[1,2-c]imidazole;
2-[4-[(Chroman-2-yl)methylamino]butyl]-1,4-dioxoperhydropyrido[1,2-a]pyrazine;
2-[4-[(Chroman-2-yl)methylamino]but-2-enyl]-1,4-dioxoperhydropyrrolo[1,2-c]imidazole;
3-[4-[2-(o-Ethoxyphenoxy)ethylamino]butyl]-2,4-dioxothiazolidine;
3-[6-[2-(o-Ethoxyphenoxy)ethylamino]hexyl]-2,4-dioxothiazolidine;
3-[8-[2-(o-Ethoxyphenoxy)ethylamino]octyl]-2,4-dioxothiazolidine;
2-[4-[2-(o-Ethoxyphenoxy)ethylamino]butyl]-1,3-dioxoperhydroimidazo[1,5-a]pyridine;
2-[6-[2-(o-Ethoxyphenoxy)ethylamino]hexyl]-1,3-dioxoperhydroimidazo[1,5-a]pyridine;
2-[4-[(2-Quinolyl)methylamino]butyl]-1,3-dioxoperhydroimidazo[1,5-a]pyridine; and
2-[6-[(2-Quinolyl)methylamino]hexyl]-1,3-dioxoperhydroimidazo[1,5-a]pyridine.

23. The use according to claim 21, wherein the isomer of the compound is selected from the group consisting of:
2-[4-[(Chroman-2(S)-yl)methylamino]butyl]-1,3-dioxoperhydropyrrolo[1,2-c]imidazole;
(E)-2-[4-[(Chroman-2-yl)methylamino]but-2-enyl]-1,3-dioxoperhydropyrrolo[1,2-c]imidazole; and
(Z)-2-[4-[(Chroman-2-yl)methylamino]but-2-enyl]-1,4-dioxoperhydropyrrolo[1,2-c]imidazole.

24. A method for preventing and/or treating a condition selected from the group consisting of cerebral damage caused by thromboembolitic stroke or traumatic brain damage, Parkinson's disease, depression, migraine, pain, psychosis, mood disorder, and urinary tract disorder in a subject in need of such prevention and/or treatment, wherein:
the method comprises administering to the subject a compound, a stereochemical isomer of the compound, or a hydrate, solvate, or pharmaceutically acceptable salt of the compound or isomer, wherein:
the compound corresponds in structure to formula I: 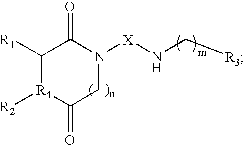
R₄ is selected from the group consisting of N and S,
if R₄ is S, then R₁ is H, and R₂ is absent;
if R₄ is N, then R₁ and R₂ are H or are methylene groups bound together forming with the heterocyclic ring a 5- or 6-membered ring;
n is zero or 1;
X is selected from the group consisting of C₂-C₁₀-alkylene, C₂-C₁₀-alkenylyne, and —CH₂—Y—CH₂—;
Y is phenyl;
m is 1 or 2;
R₃ is selected from the group consisting of chroman-2-yl, 2-quinolyl, and phenoxy, wherein:
the quinolyl, the aromatic ring of the chromanyl, and the phenyl ring of the phenoxy are optionally substituted with one or more substituents independently selected from the group consisting of C₁-C₆-alkoxy, C₁-C₆-alkyl, halogen, C₂-C₆-alkenyl, halo-(C₁-C₆)-alkyl, halo-(C₁-C₆)-alkoxy, phenyl, phenyl(C₁-C₆)-alkyl, phenoxy, C₁-C₆-alkylcarbonyl, phenylcarbonyl, phenyl(C₁-C₆)alkylcarbonyl, C₁-C₆-alkoxycarbonyl, phenyl(C₁-C₆)alkoxycarbonyl, C₁-C₆-alkyl-carbonylamino, hydroxy, cyano, nitro, amino, N—(C₁-C₆)-alkylamino, N,N—(C₁-C₆)-dialkylamino, carboxy, sulfo, sulfamoyl, sulfonylamino, (C₁-C₆)alkylaminosulfonyl, and (C₁-C₆)alkylsulfonylamino, wherein:
the C₁-C₆-alkyl portion of any of the alkyl-comprising substituents is optionally substituted with a substituent independently selected from the group consisting of hydroxy and amino; or
the phenyl ring of the phenoxy is substituted by two neighbouring residues, which together with the phenyl ring to which they are attached form tetrahydronaphthyl;
provided that the compound is not
2-[4-[(chroman-2-yl)methylamino]butyl]-1,3-dioxoperhydropyrrolo[1,2-c]imidazole,
3-[4-[(chroman-2-yl)methylamino]butyl]-2,4-dioxothiazolidine,
3-[5-[(chroman-2-yl)methylamino]pentyl]-2,4-dioxothiazolidine,
3-[6-[(chroman-2-yl)methylamino]hexyl]-2,4-dioxothiazolidine,
2-[4-[2-(phenoxy)ethylamino]butyl]-1,3-dioxoperhydropyrrolo[1,2-c]imidazole, or
3-[4-[2-(phenoxy)ethylamino]butyl]-2,4-dioxothiazolidine.

25. The method according to claim 24, wherein the subject is human.

26. The method according to claim 24, wherein the condition comprises migraine.

27. The method according to claim 24, wherein the condition comprises pain.

28. The method according to claim 24, wherein the condition comprises cerebral damage caused by thromboembolitic stroke or traumatic brain damage.

29. The method according to claim 24, wherein the condition comprises Parkinson's disease.

30. The method according to claim 24, wherein the condition comprises depression.

31. The method according to claim 24, wherein the condition comprises psychosis.

32. The method according to claim 31, wherein the psychosis comprises schizophrenia.

33. The method according to claim 24, wherein the condition comprises a mood disorder.

34. The method according to claim 33, wherein the mood disorder comprises an anxiety disorder.

35. The method according to claim 33, wherein the mood disorder comprises an aggressive disorder.

36. The method according to claim 24, wherein the condition comprises a urinary tract disorder.

37. The method according to claim 36, wherein the urinary tract disorder comprises urinary incontinence.

38. The method according to claim 24, wherein an effective amount of the compound, isomer, hydrate, solvate, or salt is administered to the subject.

39. The method according to claim 24, wherein the compound is selected from the group consisting of:
2-[4-[(Chroman-2-yl)methylamino]butyl]-1,3-dioxoperhydroimidazo[1,5-a]pyridine;
2-[4-[(Chroman-2-yl)methylamino]butyl]-1,3-dioxoperhydropyrrolo[1,2-a]pyrazine;
2-[5-[(Chroman-2-yl)methylamino]pentyl]-1,3-dioxoperhydropyrrolo[1,2-c]imidazole;
2-[6-[(Chroman-2-yl)methylamino]hexyl]-1,3-dioxoperhydropyrrolo[1,2-c]imidazole;
2-[3-[(Chroman-2-yl)methylamino]propyl]-1,3-dioxoperhydropyrrolo[1,2-c]imidazole;
3-[8-[(Chroman-2-yl)methylamino]octyl]-2,4-dioxothiazolidine;
2-[4-[(Chroman-2-yl)methylamino]butyl]-1,3-dioxoperhydropyrrolo[1,2-c]imidazole;
2-[8-[(Chroman-2-yl)methylamino]octyl]-1,3-dioxoperhydropyrrolo[1,2-c]imidazole;
2-[3-[[(Chroman-2-yl)methylamino]methyl]benzyl]-1,3-dioxoperhydropyrrolo[1,2-c]imidazole;
2-[4-[[(Chroman-2-yl)methylamino]methyl]benzyl]-1,3-dioxoperhydropyrrolo[1,2-c]imidazole;
2-[4-[(Chroman-2-yl)methylamino]but-2-enyl]-1,3-dioxoperhydropyrrolo[1,2-c]imidazole;
2-[4-[2-(o-Methoxyphenoxy)ethylamino]butyl]-1,3-dioxoperhydropyrrolo[1,2-c]imidazole;
2-[4-[2-(m-Methoxyphenoxy)ethylamino]butyl]-1,3-dioxoperhydropyrrolo[1,2-c]imidazole;
2-[4-[2-(o-Bromophenoxy)ethylamino]butyl]-1,3-dioxoperhydropyrrolo[1,2-c]imidazole;
2-[4-[2-(m-Bromophenoxy)ethylamino]butyl]-1,3-dioxoperhydropyrrolo[1,2-c]imidazole;
2-[4-[2-(o-Ethylphenoxy)ethylamino]butyl]-1,3-dioxoperhydropyrrolo[1,2-c]imidazole;
2-[4-[2-(m-Ethylphenoxy)ethylamino]butyl]-1,3-dioxoperhydropyrrolo[1,2-c]imidazole;
2-[4-[2-(o-Isopropylphenoxy)ethylamino]butyl]-1,3-dioxoperhydropyrrolo[1,2-c]imidazole;
2-[4-[(2-quinolyl)methylamino]butyl]-1,3-dioxoperhydropyrrolo[1,2-c]imidazole;
2-[4-[2-(o-Ethoxyphenoxy)ethylamino]butyl]-1,3-dioxoperhydropyrrolo[1,2-c]imidazole;
2-[4-[2-(o-Isopropoxyphenoxy)ethylamino]butyl]-1,3-dioxoperhydropyrrolo[1,2-c]imidazole;
2-[4-[2-[m-(Trifluoromethyl)phenoxy]ethylamino]butyl]-1,3-dioxoperhydropyrrolo[1,2-c]imidazole;
2-[4-[2-(1,1′-Biphenyl-2-yloxy)ethylamino]butyl]-1,3-dioxoperhydropyrrolo[1,2-c]imidazole;
2-[4-[2-[o-(Acetylamino)phenoxy]ethylamino]butyl]-1,3-dioxoperhydropyrrolo[1,2-c]imidazole;
2-[4-[2-[m-(Acetylamino)phenoxy]ethylamino]butyl]-1,3-dioxoperhydropyrrolo[1,2-c]imidazole;
2-[4-[2-[o-(Ethoxycarbonyl)phenoxy]ethylamino]butyl]-1,3-dioxoperhydropyrrolo[1,2-c]imidazole;
2-[4-[2-(5,6,7,8-Tetrahydronaphth-1-yloxy)ethylamino]butyl]-1,3-dioxoperhydropyrrolo[1,2-c]imidazole;
2-[4-[2-(2,3-Dimethylphenoxy)ethylamino]butyl]-1,3-dioxoperhydropyrrolo[1,2-c]imidazole;
2-[4-[(Chroman-2-yl)methylamino]butyl]-1,4-dioxoperhydropyrido[1,2-a]pyrazine;
2-[4-[(Chroman-2-yl)methylamino]but-2-enyl]-1,4-dioxoperhydropyrrolo[1,2-c]imidazole;
3-[4-[2-(o-Ethoxyphenoxy)ethylamino]butyl]-2,4-dioxothiazolidine;
3-[6-[2-(o-Ethoxyphenoxy)ethylamino]hexyl]-2,4-dioxothiazolidine;
3-[8-[2-(o-Ethoxyphenoxy)ethylamino]octyl]-2,4-dioxothiazolidine;
2-[4-[2-(o-Ethoxyphenoxy)ethylamino]butyl]-1,3-dioxoperhydroimidazo[1,5-a]pyridine;
2-[6-[2-(o-Ethoxyphenoxy)ethylamino]hexyl]-1,3-dioxoperhydroimidazo[1,5-a]pyridine;
2-[4-[(2-Quinolyl)methylamino]butyl]-1,3-dioxoperhydroimidazo[1,5-a]pyridine; and
2-[6-[(2-Quinolyl)methylamino]hexyl]-1,3-dioxoperhydroimidazo[1,5-a]pyridine.

40. The method according to claim 24, wherein the isomer of the compound is selected from the group consisting of:
2-[4-[(Chroman-2(S)-yl)methylamino]butyl]-1,3-dioxoperhydropyrrolo[1,2-c]imidazole;
(E)-2-[4-[(Chroman-2-yl)methylamino]but-2-enyl]-1,3-dioxoperhydropyrrolo[1,2-c]imidazole; and
(Z)-2-[4-[(Chroman-2-yl)methylamino]but-2-enyl]-1,4-dioxoperhydropyrrolo[1,2-c]imidazole.

41. The compound, isomer, hydrate, solvate, or salt according to claim 1, wherein the isomer of the compound is selected from the group consisting of:
2-[4-[(Chroman-2(S)-yl)methylamino]butyl]-1,3-dioxoperhydropyrrolo[1,2-c]imidazole;
(E)-2-[4-[(Chroman-2-yl)methylamino]but-2-enyl]-1,3-dioxoperhydropyrrolo[1,2-c]imidazole; and
(Z)-2-[4-[(Chroman-2-yl)methylamino]but-2-enyl]-1,4-dioxoperhydropyrrolo[1,2-c]imidazole.
